(19) United States
(12) Patent Application Publication
(10) Pub. No.: US 2012/0164077 A1
(43) Pub. Date: Jun. 28, 2012

(54) PREDICTIVE ASSAYS FOR CELL TRANSPLANTATION EFFICACY AND METHODS OF USING HUMAN NEUROPOTENTIATING CELLS

(76) Inventors: Tonya Bliss, Stanford, CA (US); Robert Andres, Spiegel b. Bern (CH); Gary Steinberg, Palo Alto, CA (US)

(21) Appl. No.: 13/390,439

(22) PCT Filed: Aug. 31, 2010
(86) PCT No.: PCT/US10/02377

§ 371 Date: Feb. 14, 2012

(30) Foreign Application Priority Data
Aug. 31, 2009 (US) 61272202

(51) Int. Cl. A61K 35/30; A61P 25/00; A61K 49/00; C12Q 1/02; C12N 5/079

(52) U.S. Cl. 424/9.2; 424/93.7; 435/29; 435/375

(57) ABSTRACT

The present invention provides a method of treating mammalian brain injuries, which entails administering human neuropotentiating cells to a brain of a mammal in need thereof in order to enhance dendritic and axonal plasticity, and axonal transport.

### FIELD OF THE INVENTION

The present invention relates to a method of treating mammalian brain injuries, which entails administering human neuropotentiating cells to a brain of a mammal in need thereof in order to enhance dendritic and axonal plasticity, and axonal transport. The present invention also includes in vitro assays that can be used to predict the potential efficacy of the transplanted cells in vivo.

### DESCRIPTION OF THE BACKGROUND

At present there is no proven therapy for stroke, except the thrombolytic treatments which have limited efficacy and must be administered within the first few hours after stroke. Cell transplantation offers promise as a new potential therapy for many cerebral pathologies including stroke, however, the mechanism of action of the transplanted cells is not understood. Furthermore, while stem cell transplantation promises new hope for the treatment of stroke, significant questions remain about how grafted cells elicit their effects.

Understanding how transplanted cells influence the host brain is an important step as stem cell therapies for stroke are developed as it will (a) improve the efficacy of the transplanted cells, (b) allow for identification of potential unwanted side effects, (c) allow for the determination of surrogate markers for cell efficacy that can be monitored non-invasively in patients, (d) design in vitro assays to test cell efficacy which will be critical during the cell manufacturing process, and (e) allow for the design in vivo assays to predict ceil efficacy which will be important during the pharmacological testing of the cells as the product progresses towards the clinic. However, such objectives remain to be attained.

### SUMMARY OF THE INVENTION

The present invention is based, in part, upon the discovery of parameters that can be tested both in vitro and in vivo, that allow for predicting the efficacy of transplanted human neuropotentiating cells, such as neural progenitor cells (hNPCs), to enhance recovery after various brain injuries including stroke. These parameters include dendritic plasticity, axonal plasticity, and axonal transport, all of which are enhanced by hNPC transplantation after stroke and coincide with enhanced functional recovery. The present invention, thus, provides predictive assays for cell transplantation efficacy and methods of using human neuropotentiating cells.

More specifically, the present inventors demonstrate that axonal transport, which is critical for both proper axonal function and axonal sprouting, is inhibited by stroke and that rescue thereof is effected by hNPC treatment. Additionally, the present invention provides in vitro co-culture assays in which hNPCs can mimic these effects observed in vivo.

Accordingly, the present invention also provides a method of enhancing dendritic- and axonal plasticity, as well as axonal transport in order to promote post-ischemic recovery.

### BRIEF DESCRIPTION OF THE DRAWINGS

FIG. 1 illustrates an experimental design used in the present invention, including an experimental timeline.

FIG. 2 illustrates the preparation of human neural progenitor cells (hNPCs).

FIG. 3 illustrates differentiation fate of transplanted hNPCs and effect on behavioral recovery after stroke.

FIG. 4 illustrates dendritic plasticity of the brain after stroke.

FIG. 5 illustrates hNPC transplantation enhances dendritic plasticity after stroke.

FIG. 6 illustrates axonal plasticity after stroke.

FIG. 7 illustrates hNPCs enhance axonal sprouting after stroke.

FIG. 8 illustrates hNPCs enhance GAP-43 expression at 5 weeks post-transplantation.

FIG. 9 illustrates in vitro assay for identification of secreted factors mediating hNPC-induced dendritic and axonal plasticity.

FIG. 10 illustrates thrombospondins 1/2 required for axonal sprouting and functional recovery after stroke.

FIG. 11 illustrates parameters influenced by VEGF.

FIG. 12 illustrates that SPARC and Slit can influence neurite outgrowth.

FIG. 13 illustrates identification of secreted factors mediating neurite plasticity in vitro.

FIG. 14 illustrates effects of hNPCs on anterograde axonal transport in vivo and in vitro.

FIG. 15 illustrates frequency class analysis of vesicle transport.

### DETAILED DESCRIPTION OF THE PREFERRED EMBODIMENTS

Term Definitions:

fMRI—functional magnetic resonance imaging-is a type of specialized magnetic resonance imaging that measures hemodynamic response related to neural activity in the brain or spinal cord of humans or other mammals.

Neuropotentiating—as used herein, the term means generally cells having the potential to become neurons, or at least cells that have the potential to enhance plasticity of existing neurons. Examples are mesenchymal stem cells (MSC) and hNPCs. Others are neurons, presently made from fibroblasts, i.e., induced neurons; blood-, bone marrow- and adipose tissue-derived cells as well as olfactory ensheathing cells.

Slit proteins-referred to as ‘Slit’, refers to a type of protein identified as a ligand for the repulsive guidance receptor Robo. These proteins are believed to have an evolutionarily conserved role in axon guidance, and determine the dorsoventral position of major axonal pathways in the mammalian brain. The known human Slit proteins contain from 1523 to 1534 amino acids. Examples are human Slit-1 and Slit-2.

The present invention is based, in part, on the recognition by the present inventors that bilateral reorganization of surviving circuits after cerebral ischemia is associated with recovery after stroke. The present inventors have discovered that neuropotentiating cells, such as hNPC (human neural progenitor cells) can enhance dendritic plasticity in both the ipsi- and contralesional cortex and this coincides with hNPC-induced functional recovery.

The present inventors also demonstrate herein that hNPC-grafted rats demonstrated increased cortico-cortical, cortico-striatal, cortico-thalamic and cortico-spinal axonal rewiring from the contralesional side; both the transcollosal cortico-spinal axonal sprouting correlated with functional recovery.

The present inventors also demonstrate herein that axonal transport, which is critical for both proper axonal function and axonal sprouting, is inhibited by stroke and that this is rescued by hNPC treatment. Importantly, this identifies another likely mechanism of action of transplanted cells.

The present inventors also provide herein in vitro co-culture assays in which hNPCs can mimic the effects observed in vivo. More specifically, through immunodepletion studies, the present inventors identified vascular endothelial growth factor (VEGF), thrombospondins (TSP) 1 and 2, and Slit as mediators, at least, partially responsible for hNPC-induced effects on dendritic and axonal plasticity in vitro. VEGF was also demonstrated to mediate effects on axonal transport.

Thus, in accordance with the present invention, hNPCs are considered to aid recovery after stroke through secretion of factors that enhance brain repair and plasticity.

The present invention, thus, provides many aspects relating to the Investigation and treatment of strokes, particularly cerebral ischemia.

First, a method of assessing efficacy of transplanted hNPCs in promoting recovery from brain injury or surgery is provided.

Second, a method of assessing efficacy of transplanted hNPCs in promoting recovery from spinal injury or surgery is provided.

Third, a method of evaluating mechanisms for developing axonal plasticity is provided.

Fourth, a method of determining pharmacological activity of transplanted cells in a mammal, particularly a human, is provided.

Fifth, a method of simulating in vivo effects of hNPCs in a cell culture assay is provided.

Sixth, a method of treating brain injury in a mammal, particularly a human, is provided.

Seventh, a method of promoting recovery from brain or spinal surgery in a mammal, particularly a human, is provided.

Eighth, genetically-engineered cells which secrete elevated amounts of one or more substances which improve brain or spinal plasticity is provided.

Other aspects are also included, which will be described below in more detail using hNPCs as an example.

Methods: (a) In vivo: hNPCs or buffer were transplanted into the ischemic cortex of NIH Nude rats 7 days after stroke induction using the distal middle cerebral artery Occlusion (dMCAo) stroke model. Rats were subjected to a battery of behavior tests starting before stroke to get a baseline, at 7 days after stroke (prior to transplantation) and weekly after transplantation for 4 weeks. To visualize dendrites, brain slices were stained using the Rapid Golgi stain. Dendritic branching, total dendritic length, and branch order were measured in layer V of the ipsilesional and contralesional cortex at 2 and 4 weeks post-transplantation.

To measure axonal sprouting, animals were injected with the anterograde axonal tracer biotinylated dextran (BDA) into the contralesional cortex at 2 or 4 weeks post-transplantation and sacrificed 1 week thereafter. The extent of axonal sprouting towards the lesioned cortex and the corticospinal tract was quantified in different brain regions by confocal image acquisition and Image J analysis. To determine axonal transport, brain slices were stained for the APP protein which accumulates when axonal transport is inhibited.

(b) In vitro: To investigate the underlying mechanisms of axonal plasticity, hNPCs were indirectly co-cultured with rat E14 cortical or striatal progenitor cells during days 3-7 in vitro, and then stained for neurofilaments (SMI312 antibody) to label axons, or MAP2 to label dendrites. Immunodepletion studies were carried out with either neutralizing antibodies against vascular endothelial growth factor (VEGF), thrombospondin (TSP)-1/2, and SPARC, or Slit-neutralizing ROBO-Fc chimeras. Species-matched antibody isotypes were used as controls. Axonal and dendritic outgrowth were quantified using automated high-throughput image analysis software. Effects on the rate of anterograde axonal transport were assessed by live imaging of dextran-labeled vesicles in cultured rat cortical neurons using a compartmentalized microfluidic culture platform with hNPCs or vehicle added to the reservoir distal to the axonal compartment.

Results: hNPC-treated animals exhibited significant enhanced functional recovery on 3 out of the 4 behavior tests starling as early as 2 weeks post-transplantation. hNPCs enhanced dendritic plasticity after stroke in both the ipsilesional and contralesional cortex. At 2 weeks post transplantation, dendritic branching (db) and total dendritic length (dl) were significantly increased in the hNPC group (compared to the buffer group) in the contralesional (p=0.001 (db) p=0.003 (dl)) and ipsilesional (p_{<}0.001 (db) p=0.01 (dl)) hemispheres. At 4 weeks, similar increases (p<0 05) in dendritic branching and length were only observed in the ipsilesional hemisphere. Changes in dendritic branching were mostly due to an increase in the number of 3^{rd} and 4^{th} order branches. hNPC-grafted rats also showed significantly greater extension of BDA-labeled host axons from the contralateral (uninjured) hemisphere towards the damage hemisphere including cortex, striatum, thalamus and the ipsilateral corticospinal tract after dMCAO than controls (p<0.05). Staining for neurofilaments (SMI312 antibody) further revealed a higher density of axons in the corpus callosum of hNPC-grafted rats than in controls (p<0.05). In addition, the post-ischemic increase of APP-positive axonal puncta observed in the ipsi- and contralesional corpus callosum was significantly attenuated in hNPC-treated animals, as compared to controls (p<0 05), indicating that hNPCs help restore axonal transport after stroke. hNPCs were also found to enhance the aforementioned parameters in vitro. Co-culture of cortical and striatal progenitor cells with hNPCs resulted in enhanced axonal outgrowth, dendritic length, dendritic branching, and axonal transport after 5 days in culture (p<0.01). The effects on axonal and dendritic outgrowth were partially abolished when VEGF, TSP-1/2, or Slit, but not SPARC, were neutralized (p<0.01).

Transplanted hNPCs significantly enhance structural plasticity and axonal transport after stroke in vivo, and this coincides with enhanced functional recovery after stroke in the hNPC treated group. Similar changes in neurite structural plasticity and axonal transport were induced by hNPCs in vitro. VEGF, TSP-1/2, and Slit were identified as at least partially responsible mediators of the effects on axonal and dendritic outgrowth in vitro. Understanding how hNPCs augment host brain plasticity allows for and enables treatments for stroke and other disorders of the nervous system, including, for example, the brain and spinal system.

The following important conclusions are made based upon experiments conducted.
1. Our results indicate various parameters to investigate when determining the pharmacological activity of transplanted cells in animals. These parameters would be in addition to the usual end point assay showing that the cells improve functional outcome.
2. Our results indicate that neuropotentiating cells, such as hNPCs, for example, enhance axonal sprouting (and transport) after stroke. Changes in such axonal properties can be monitored non-invasively in patients using diffusion tensor imaging (DTI). Therefore, the present invention has important clinical implications as it provides a surrogate marker of stem cell efficacy that can be monitored in patients. This is important for use of cell therapy in the clinic.
3. The present invention provides a means to mimic the in vivo effects of hNPCs, for example, in cell culture assay. This in vitro assay to determine the effect of hNPCs, for example, on neurite (axon+dendrites) outgrowth, and axonal transport will be useful during the cell manufacturing process. It offers a relatively high throughput assay to screen hNPCs at each stage of the manufacturing process this will help confirm that the cell product retains activity that is potentially pertinent to its in vivo efficacy.
4. High throughput bioassays for hNPC, for example, are an essential component to test cell activity at different stages during the cell manufacturing process to obtain active GMP grade cells for clinical use.
5. Various assays may be used to either identify hNPCs or other non-neural cells, such as MSCs, to characterize these cells in order to grade them by their ability to secrete various neuropotentiating substances, i.e., have the potential to enhance plasticity of neurons. For example, an hNPC identifying assay as disclosed in U.S. Pat. No. 7,442,545 may be used. Further, a VEGF assay as disclosed in U.S. Pat. No. 6,787,323 may be used to determine VEGF bioactivity of hNPCs. Both of these patents are incorporated herein by reference in the entirety.

Generally, the clinical treatment methods of the present invention include, for example, a method of treating brain injury, a method of treating spinal injury, a method of promoting recovery after brain or spinal surgery, and a method of measuring axonal properties in patients after stroke or surgery upon and after transplantation of hNPCs or other neuropotentiating cells.

hNPCs, engineered hNPCs or other cells with neuropotentiating ability are cultured, harvested and then transplanted into a patient. Generally, the cells are transplanted into at least the ipsilesional cortex and tests are administered to establish a baseline for the patient. This may be done non-invasively by diffusion tensor imaging (DTI). Follow up DTI testing is generally conducted at post-transplantation intervals, such as 2 and 4 weeks, for example.

Diffusion tensor imaging (DTI) is used in imaging and analysis of brain pathologies. For example, U.S. Pat. No. 6,463,315 describes analysis of cerebral white matter for prognosis and diagnosis of neurological disorders. Such techniques have been refined as described in U.S. Pat. No. 7,355,403 for noise reduction in diffusion tensor imaging data using Bayesian methods. Both patents are incorporated herein by reference in the entirety. These methodologies may be used in accordance with the present invention in combination with the routine skill of the artisan.

Generally, any type of cell that confers neuropotentiating properties may be used. By ‘neuropotentiating’ is meant an ability to enhance neural functioning, which may include enhanced dendritic plasticity, axonal branching and axonal transport. Examples of cells which may be used in accordance with the present invention include hNPCs obtained from many sources, such as fetal brain tissue, adult brain tissue, human embryonic stem cell, somatic cells like skin fibroblasts that are made into IPS (induced pluripotent stem) cells that are embryonic-like stem cells, and olfactory ensheathing cells.

Further, for brain injury or recovery after brain surgery, exemplary sites in the brain for transplantation include, but are not limited to, the ipsilesional cortex or the striatum/basal ganglia.

Generally, the following procedure is followed:
1) A CT scan of a patient's head is conducted to find the location, volume and geometry of the lesion resulting from a stroke or from another injury to the brain;
2) From the CT image obtained, transplant trajectories are determined with the objective in mind of introducing the injected cells into or proximal to the lesion. Hence, the injection sites encompass the lesional region;
3) Generally, for intracerebral delivery, between about 5 to 50 million cells per patient are used, and which are spread out among about 5 to 75 sites, more preferably from about 25 to 50 sites. For injection into the periphery (into blood), about from 100 to 800 million cells are used.

Generally, the neuropotentiating cells are introduced by intravenous-, intra-arterial and/or intracerebral injection.

Additionally, while at least about 5,000 cells are transplanted per site, as many as several million per site may be transplanted. Moreover, from 1 to about 100 injection or transplantation sites may be used per patient, which may be any mammal, but particularly a human. Generally, from about 25 to 50 sites are used.

The drawings will be described below in more detail.

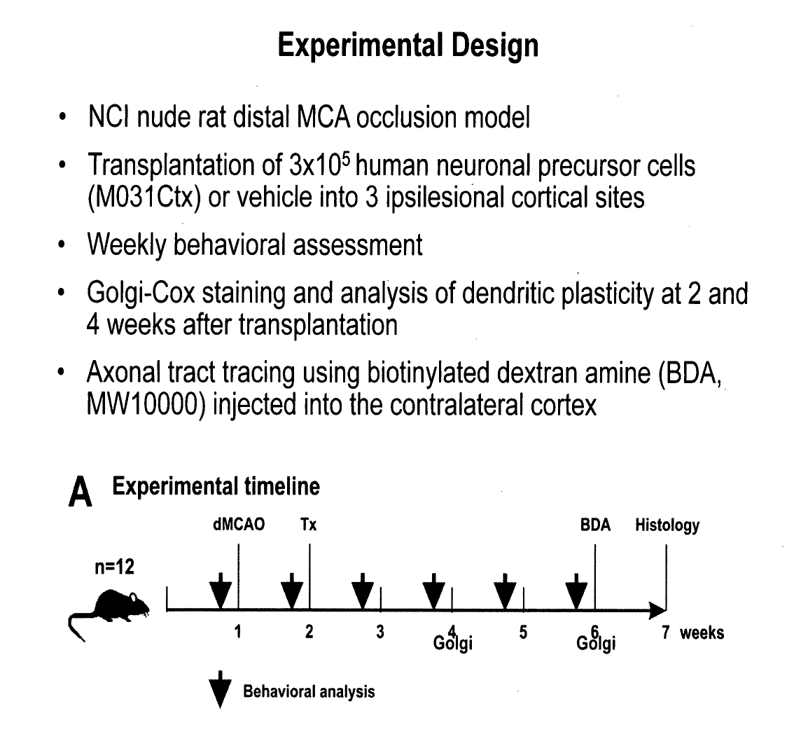

FIG. 1 illustrates an experimental design used in accordance with the present invention with transplantation of hNPCs into 3 ipsilesional cortical sites with about 100,000 cells per site. Weekly behavioral assessment was conducted. Golgi staining was conducted followed by analysis of dendritic plasticity at 2 and 4 weeks post-translation. Axonal tract tracing was effected with biotinylated dextran amine (BDA) as described.

Generally, transplantations will involve from about 5,000 to 100,000 hNPCs in at least one cortical site. More generally, at least such amounts are transplanted to at least two cortical sites, preferably in the ipsilesional cortex.

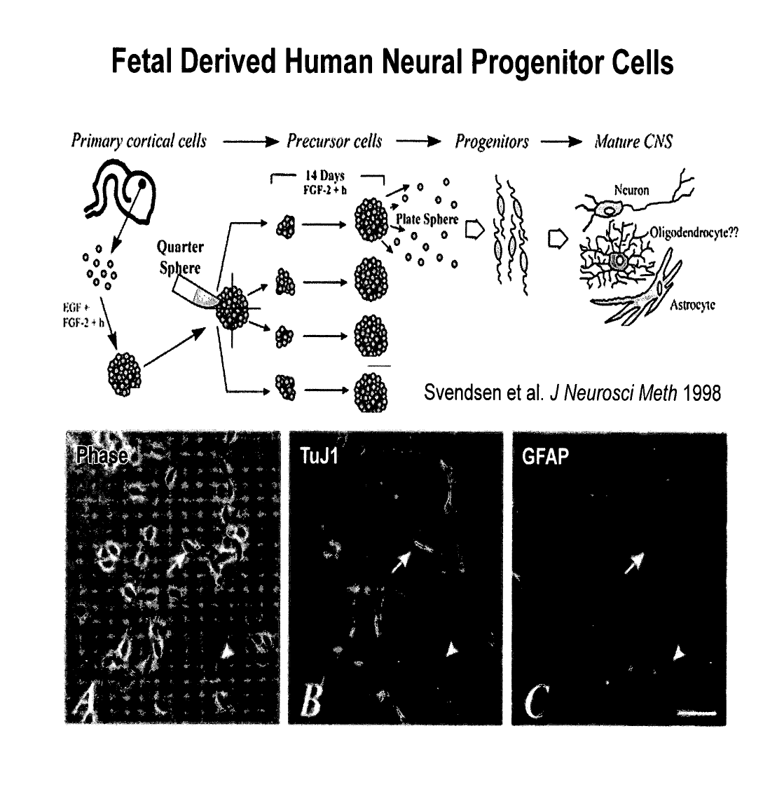

FIG. 2 illustrates the generation of hNPCs from primary cortical cells through precursor cells to progenitor cells followed by CNS integration. This development is shown in the progression from insets A>B>C.

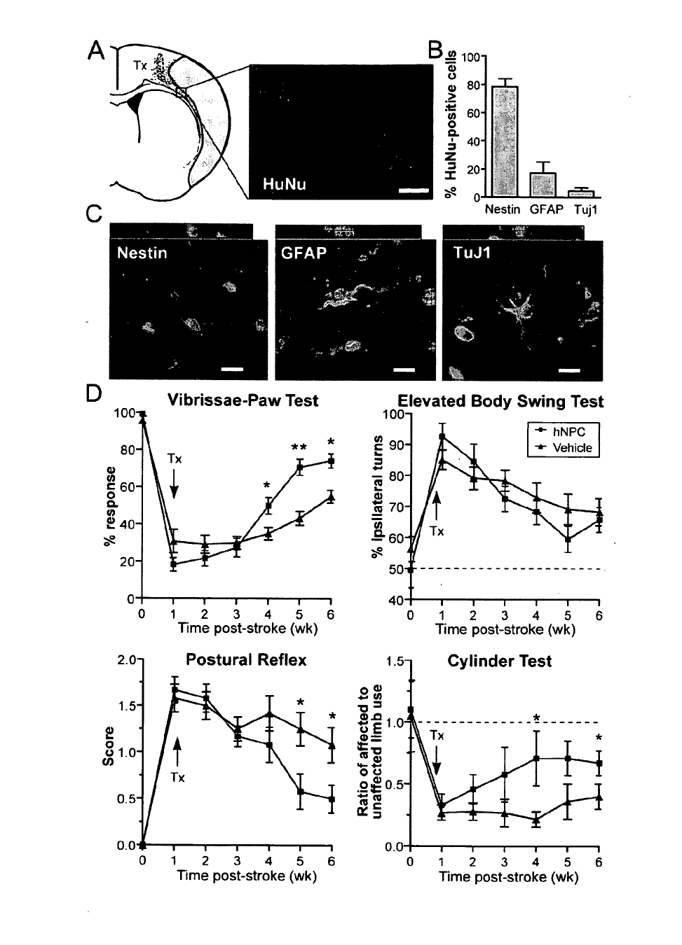

FIG. 3 illustrates differentiation fate of transplanted hNPCs and effect on behavioral recovery after stroke.

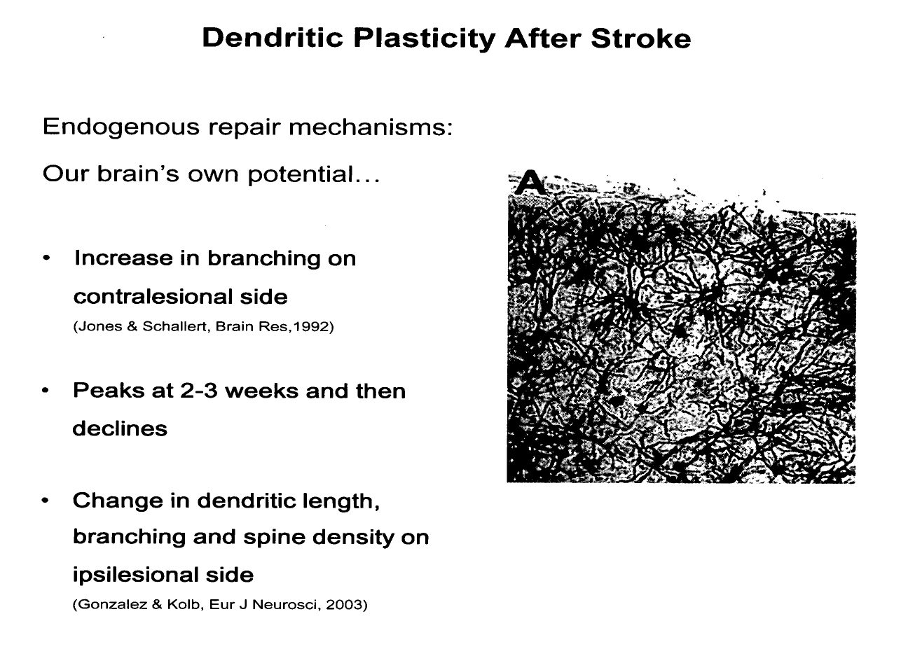

FIG. 4 illustrates dendritic plasticity of the brain after stroke.

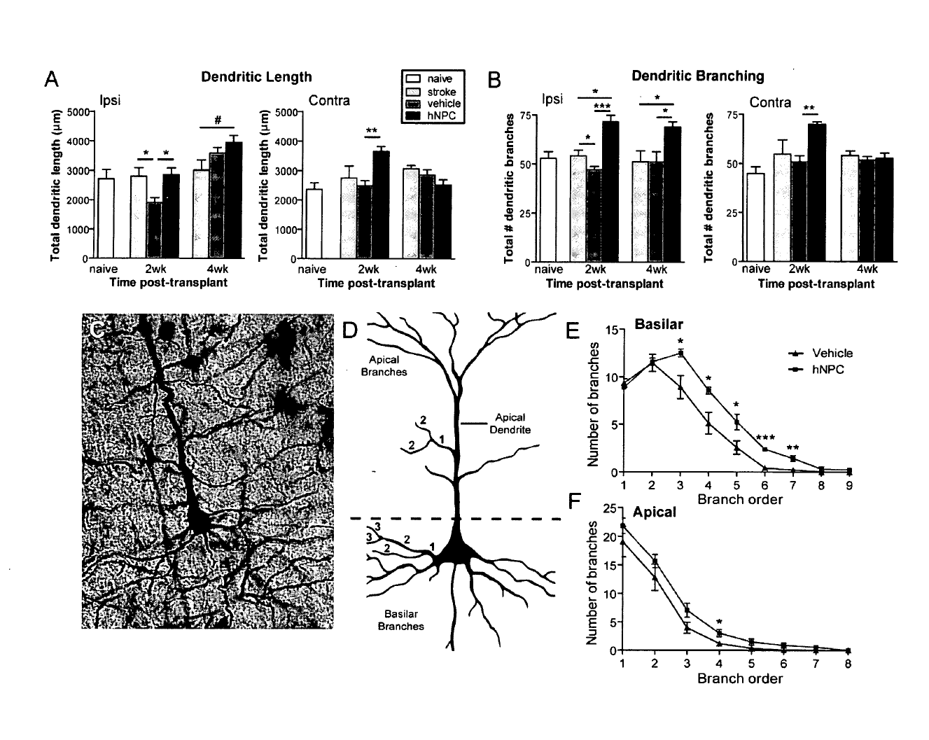

FIG. 5 illustrates hNPC transplantation enhances dendritic plasticity after stroke

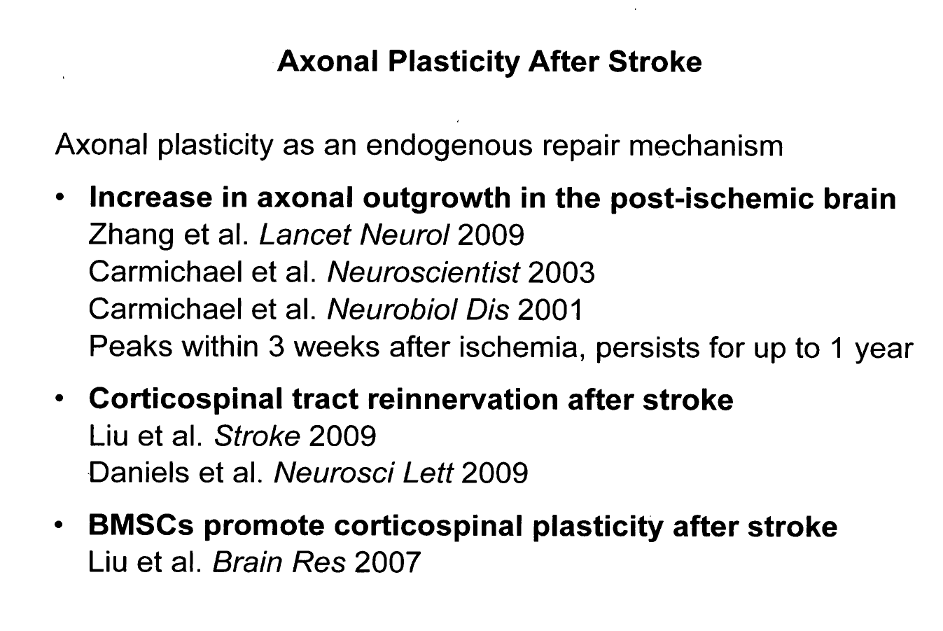

FIG. 6 illustrates axonal plasticity after stroke.

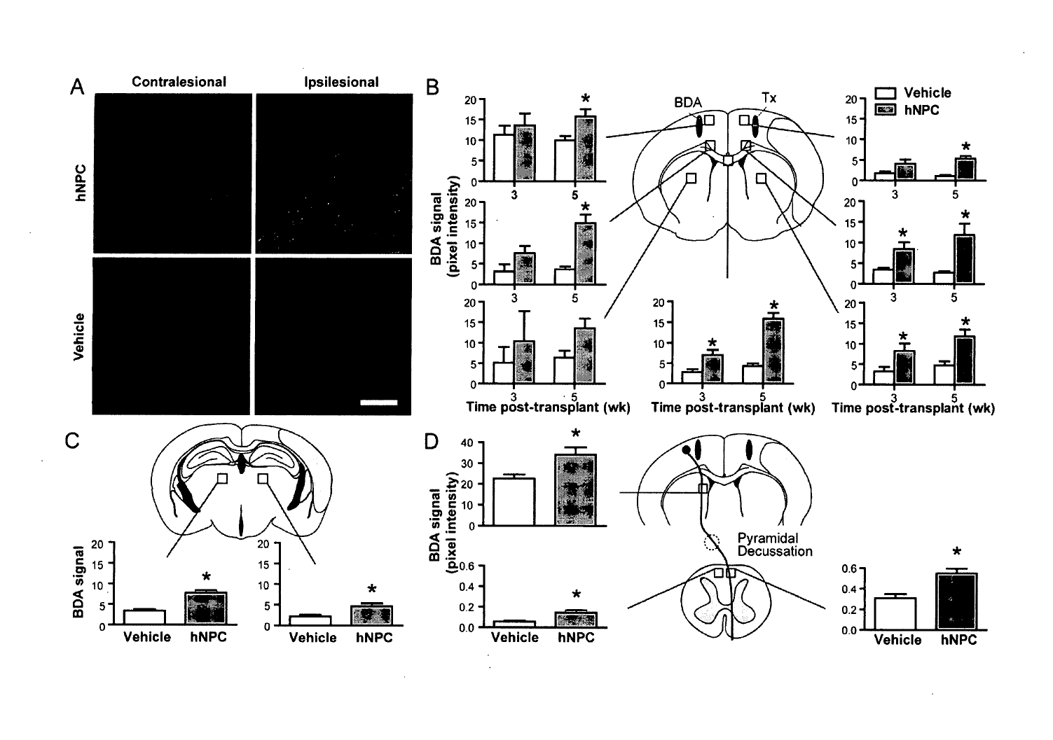

FIG. 7 illustrates hNPCs enhance axonal sprouting post-stroke.

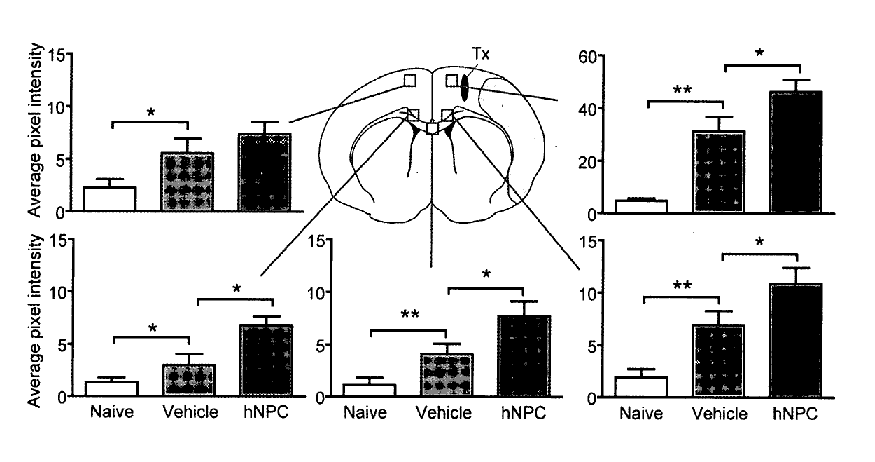

FIG. 8 illustrates hNPCs enhance GAP-43 expression at 5 weeks post-transplantation.

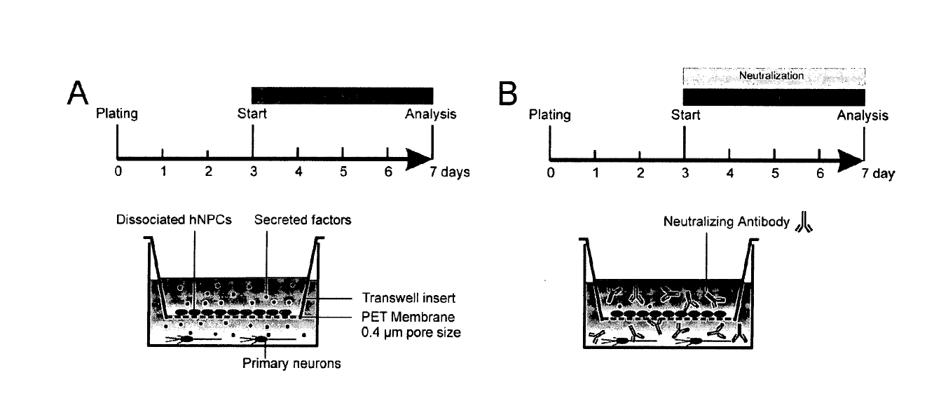

FIG. 9 illustrates in vitro assay for identification of secreted factors mediating hNPC-induced dendritic and axonal plasticity.

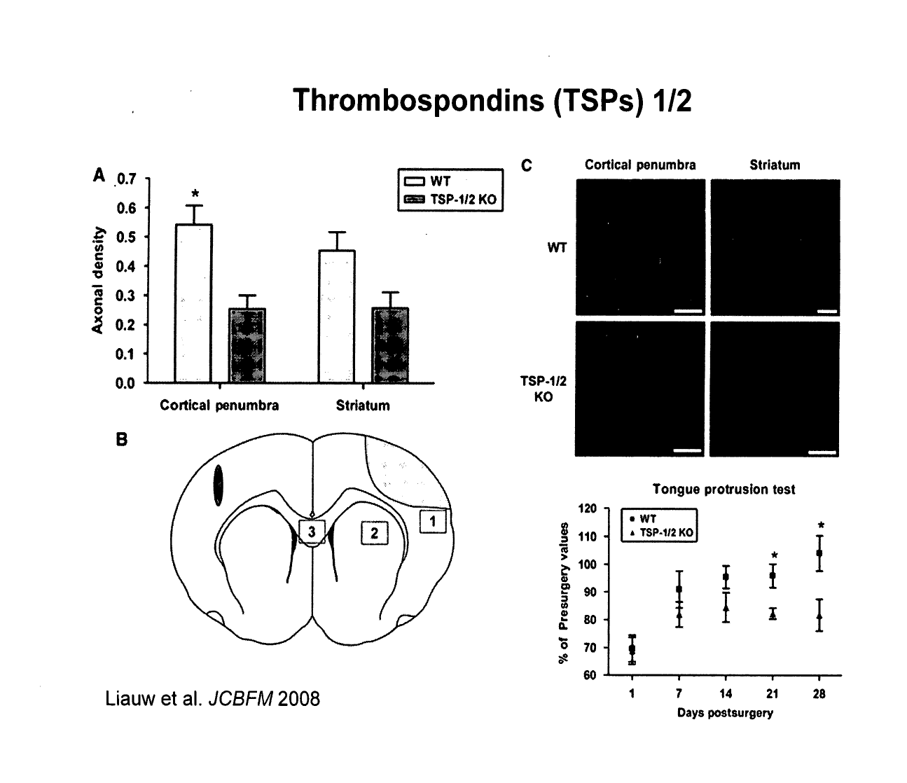

FIG. 10 illustrates thrombospondins 1/2 required for axonal sprouting and functional recovery after stroke.

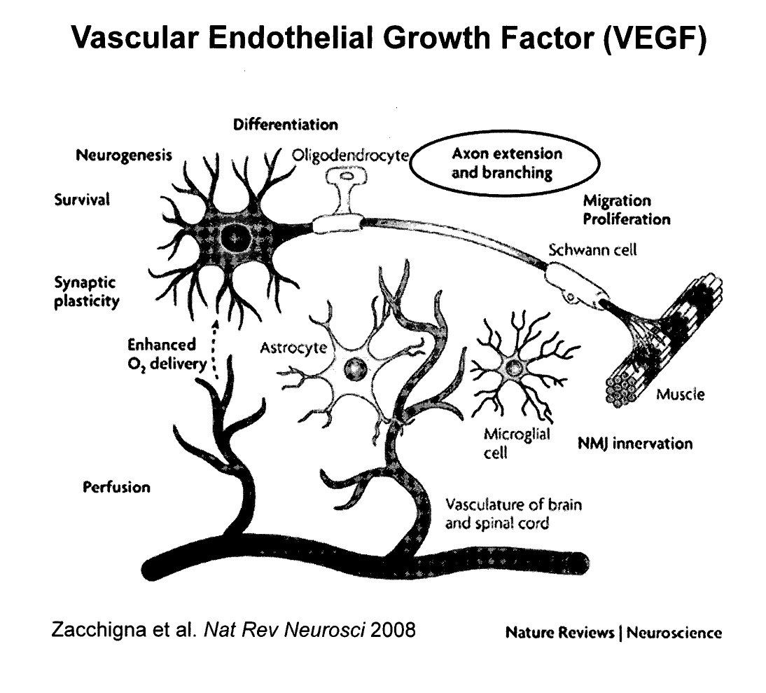

FIG. 11 illustrates parameters influenced by VEGF.

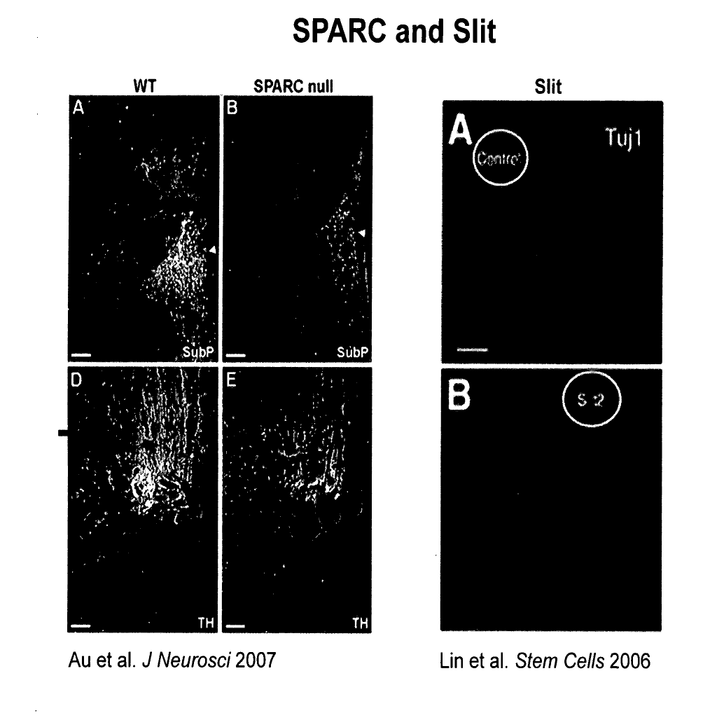

FIG. 12 illustrates that SPARC and Slit can influence neurite outgrowth.

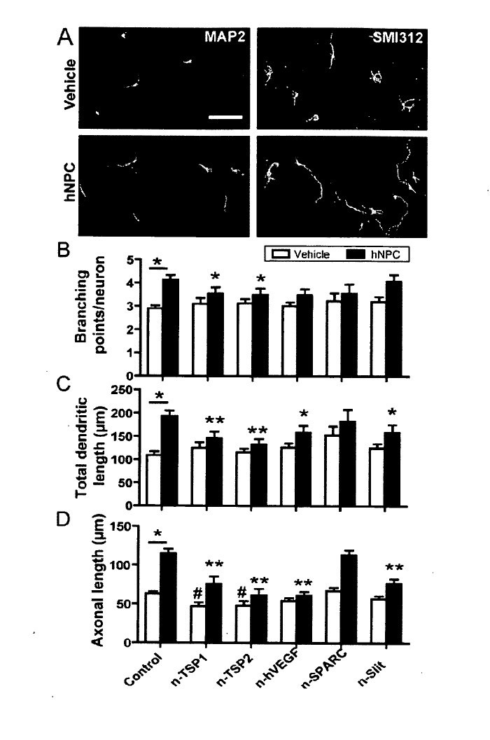

FIG. 13 illustrates identification of secreted factors mediating neurite plasticity in vitro.

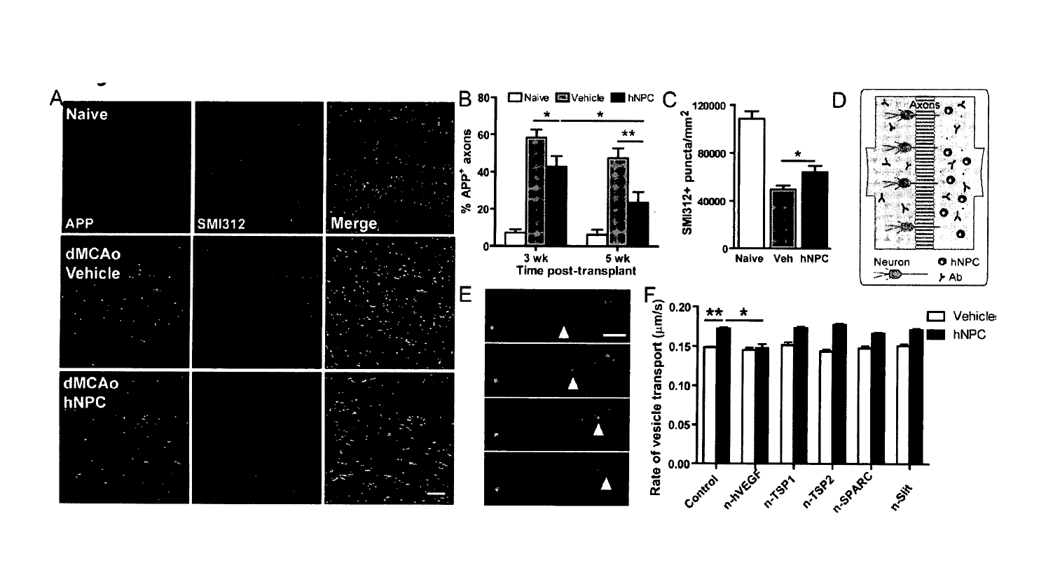

FIG. 14 illustrates effects of hNPCs on anterograde axonal transport in vivo and in vitro.

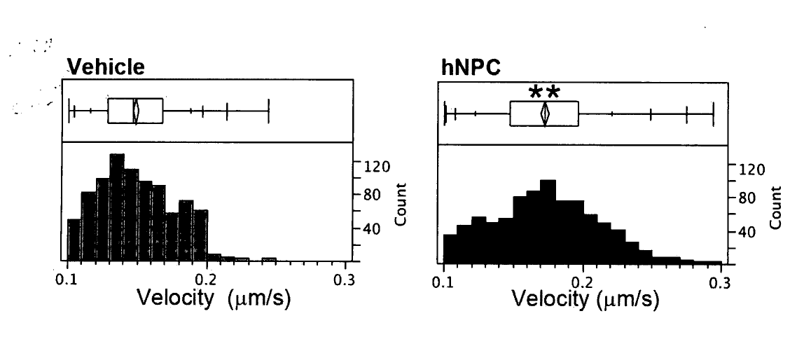

FIG. 15 illustrates frequency class analysis of vesicle transport.

### Stem Cells: General Discussion pf Transplantation Considerations

The advent of stem cells offers an exciting new therapeutic avenue for stroke not only to prevent damage, which has been the focus of conventional therapeutic strategies, but also to actually repair the injured brain. Cell transplantation has shown much promise in experimental models of stroke with a diverse array of cell types including brain-, bone marrow-, and blood-derived progenitors reported to enhance functional recovery after ischemic and hemorrhagic stroke. Such results led to early Phase I and II clinical trials (see Table 1 below) using either a cell line of immature neurons (hNT) derived from a human teratocarcinoma, fetal porcine cells, or autologous mesenchymal stem cells (MSCs). These studies focused on the safety and feasibility of cell transplantation therapy. No cell-related adverse effects were reported with the hNT and MSC transplants. However, 2 of the 5 patients receiving the xenotransplant either developed seizures or aggravation of motor deficits and the contribution of the cell therapy to these adverse effects was unclear. Conclusions about efficacy of the different treatments are difficult to draw due to small sample sizes for each trial but, overall, treatment did not worsen recovery and at best showed slightly improved outcomes.

### Therapeutic Time Window

A major promise of cell therapy, now also amply demonstrated by the present invention, is that it will open the therapeutic time window of intervention, thus benefiting a significantly larger patient population. The literature reports a wide range of successful stroke-to-transplantation intervals. The majority of pre-clinical studies have transplanted within the first 3 days after stroke and these have mostly used bone marrow- or blood-derived cells. This time window is already greater than the 3-6 h window required for t-PA therapy, the only treatment for stroke that currently exists. Cell enhanced recovery has also been reported with sub-acute (1 week post-stroke) and chronic (>3 weeks post-stroke) delivery of many cell types including neural cells .However, comparison of the results to identify an optimum time for transplantation is difficult as the studies used different models of stroke, cell types, methods of cell delivery, and behavioral tests to assess efficacy. This highlights the need for a more methodical and standardized approach to pre-clinical research so that direct comparisons can be made between individual studies. The present invention provides this.

Generally, the preferred time for transplantation is dependent on the cell type used and their mechanism of action. If a treatment strategy focuses on neuroprotective mechanisms, acute delivery is important. If the cells act to enhance endogenous repair mechanisms (e.g. plasticity and angiogenesis), then sub-acute delivery is pertinent as these events are demonstrably more prevalent in the first few, weeks after ischemia. The route of delivery (discussed later) may also dictate the timing of transplantation. Intravascular transplantation may require early administration as the cells use inflammatory signals to home to the injured brain, although MSCs were also found in the brain after late intravascular delivery (1 month post-stroke). In contrast, intraparenchymal injection of cells benefit from later delivery once the initial inflammatory response has subsided, as this affords greater cell survival.

All of the aforementioned clinical trials opted for delivery of cells in the post-acute phase of stroke (see Table 1 below). Importantly, the experiments demonstrate that the delivery of cells at different times is feasible.

### Patient Selection

### Patient Age and Sex

Stroke is a heterogeneous disease that typically affects elderly patients with significant comorbidities such as atherosclerosis, hypertension and diabetes mellitus. In addition, men and women have different risk factors for stroke, exhibit different stroke pathologies, and respond differently to treatment. However, most pre-clinical studies have been performed in healthy, young adult, male laboratory animals, which fail to represent the complex human pathology. For example, a body of work from the Chopp group implies that MSC transplantation aids recovery in part by modulating the host astrocytic response to stroke, yet there are marked sex differences in how astrocytes respond after stroke, which raises the question of whether MSCs would have the same efficacy in male and female rats. Similar issues pertain to the response of the aged brain to stroke; aged rats showed higher astrocyte reactivity, increased macrophage recruitment, and delayed neuronal death after hemorrhagic stroke, as compared to younger animals. Moreover, the extent of ischemic damage and blood-brain barrier breakdown increased with aging in female mice, whereas male animals showed opposite effects. In addition, in establishing clinical trials, cell dosage becomes an important balance of efficacy and tolerance; these parameters may also have sex and age-related differences which must be taken into consideration when designing pre-clinical studies.

### Lesion Location and Size

Lesion location and size are important factors in determining patient suitability for cell therapy. The majority of pre-clinical studies show cell-enhanced recovery after striatal lesions although cell-induced improvements with cortical lesions are also reported However, not all studies find that cell therapy is effective. Two groups report that neural progenitor cells (NPCs) improve recovery, but only if combined with enriched housing, and we have found very little effect of hNT cells in cortical stroke despite multiple studies showing efficacy of the same cells with striatal stroke. Similarly, Makinen et al found no behavioral improvement after transplantation of human umbilical cord blood stem cells while other studies using similar cells, stroke model, and timing of transplantation did report recovery. Such ‘negative’ data, which are often not published, are important to consider, as they will help define the preferred conditions for cell transplant therapy. For example, do ‘negative’ studies use different behavior tests, or perhaps include animals with larger lesions while ‘positive’ studies exclude animals with large or very small lesions. More discussion of inclusion/exclusion criteria is required in the field. ‘Negative’ studies also highlight the need for a collaborative effort among multiple laboratories to confirm the efficacy of a particular stem/progenitor cell using the same study parameters (stroke model, timing of transplantation, behavior tests, rodent strain, age and gender). Despite being expensive and very labor intensive, these confirmative studies bring an unprecedented degree of veracity that is essential for translation of cell therapy to the clinics

### Ischemic Versus Hemorrhagic Stroke

The pathophysiology and mechanisms of recovery differ between ischemic and hemorrhagic strokes. For example, there is no salvageable penumbra with intracerebral hemorrhage (ICH) unlike ischemic stroke, and patients with ICH do not suffer from reperfusion injury with its burst of free radical production. Toxic blood breakdown products like thrombin, hemoglobin, and iron additionally contribute to neuronal damage after ICH. Therefore, it is plausible that hemorrhagic and ischemic stroke may respond differently to cell therapy and should be tested separately in clinical trials.

### Route of Cell Delivery

Functional recovery has been reported with intracerebral, intravascular, and intracerebroventricular delivery of cells but the best route is not apparent. Intracerebral delivery results in more transplanted cells in the brain targeting the lesion compared to other delivery routes. It is speculated however, that intravascular delivery may be more appropriate for larger lesions as it could lead to wider distribution of cells around the ischemic area. Generally, cells need to be near the lesion to be effective, i.e., at least proximate thereto.

Each route of delivery has safety issues. Intravascular delivery is less invasive than injection into the brain but raises concern of cells sticking together creating microemboli, and cells homing to other organs. Intraarterial (intracarotid) administration is preferable to intravenous infusion, allowing first-pass delivery resulting in better targeting of cells to the brain and fewer cells found in other organs. However, Bang et al reported no adverse effects of intravenous infusion of MSCs in their clinical trial. Intraparenchymal transplantation avoids this biodistribution issue, but is more invasive and often results in a physical mass of cells which itself could disrupt the healthy tissue. However, no detrimental effects were reported in the Phase I and II clinical trials with hNT cells that were injected into several striatal sites surrounding the lesion. Transplantation into the lesion cavity may also be used. This requires cells to either be encapsulated or delivered within a scaffold to facilitate their survival in such a hostile inflammatory environment that lacks trophic support. In using this strategy, the biocompatibility of the matrix material with the patient and the transplanted cells must be considered. In addition to the above considerations, several guidelines may be set forth for cell delivery in accordance with the present invention.

### Cell Type and Source

A variety of human cell types may be used in the present invention, such as: (1) neural stem/progenitor cells; (2) immortalized cell lines; and (3) hematopoietic/endothelial progenitors and stromal cells isolated from bone marrow, umbilical cord blood, peripheral blood, or adipose tissue. To become a useful therapeutic option, cells must show efficacy, have a large expansion capacity in culture to meet the eventual clinical demand, and must meet strict criteria for stability and safety. The present inventors have addressed this with respect to the different cell types below.

### (1) Neural Progenitor Cells

NPCs have the potential to become neurons, astrocytes and oligodendrocytes, which might be advantageous given that stroke injury damages all three cell types. However, the involvement of cell replacement in functional recovery remains under investigation (discussed in the Mechanism section below). NPCs can be derived from several sources.

(a) Human Embryonic stem cell (hES)-derived NPCs: hES cells can be differentiated into NPCs by various methods (Daadi et al., 2008; Koch et al., 2009; Reubinoff et al., 2001; Studer, 2001; Zhang et al., 2001). Whether different protocols, or even if different hES lines result in distinguishable populations of NPCs is not understood, but enhanced recovery after transplantation into the stroke brain has been reported by several groups using different preparations of hES-derived NPCs (Daadi et al., 2008; Hicks et al., 2009; Ikeda et al., 2005; Theus et al., 2008). Moreover, integration into the host brain has also been reported (Buhnemann et al., 2006; Hayashi et al., 2006; Daadi et al., 2009a). Although there are ethical concerns regarding the use of hES cells, these may eventually be overcome by the use of IPS cells (induced pluripotent stem cells), whereby somatic cells such as fibroblasts can be reprogrammed to become ES-like cells by the addition of 3 or 4 critical factors (Park et al., 2008; Takahashi et al., 2007).

An advantage of hES cells is their capacity to propagate in culture over many passages providing a virtually unlimited supply of NPCs, which is necessary for clinical application. However, batch-to-batch variations in the resultant NPCs must be considered. The price of this proliferative capacity is the tendency of hES cells to form tumors (Carson et al., 2006) and it is imperative that undifferentiated hES cells are removed from NPC preparations. Proof that hES-derived cells are safe may prove difficult in pre-clinical studies as Erdo et al (Erdo et al., 2003) showed that xenografts are less tumorigenic than allografts. Therefore, the true tumorigenic potential of human cells may not be realized until they are tested in patients. Yet, despite these concerns, Geron has recently received FDA approval to use hES-derived NPCs in a clinical trial for acute spinal cord injury.

(b) Fetal-derived NPCs: The first clinical trial (Phase I) using fetal human NPCs was recently completed for Batten disease, a CNS lysosomal storage disease (Taupin, 2006). Although the results have not been published, it's been informally reported that there were no adverse effects related to transplantation of the human NPCs and autopsy of a patient who died of the disease showed the injected cells engraft and survive in the brain for close to a year. We used similar cells in a cortical stroke model and found good survival and migration towards the lesion (Kelly et al., 2004), and recently demonstrated improved functional recovery with these cells (unpublished data). Ishibishi et al (Ishibishi et al., 2004) also found functional recovery with fetal derived NPCs. The tumorigenic potential of fetal-derived NPCs is less than hES-derived cells, although thorough characterization of the cells is vital (Amariglio et al., 2009). A consideration, however, is the limited in vitro expansion potential of fetal-derived cells, which may be problematic for adequate clinical supply.

### (2) Cell Lines

Several human neural cell lines are reported to elicit functional recovery after stroke (Borlongan et al., 1998; Chu et al., 2004; Jeong et al., 2003; Stroemer et al., 2008). These cell lines are immortalized, either because they originate from a teratocarcinoma (such as the hNT neurons) (Andrews et al., 1984, Newman et al., 2005), or because they are transformed with an oncogene like myc; this is the case for the human fetal neural stem cell line ReN001 from ReNeuron (Stroemer et al., 2008) which is currently being considered for stroke clinical trials in the UK (www.reneuron.com). Being immortalized, these cell lines have the advantage of potentially unlimited expansion in culture. However, the risk of malignant transformation of immortalized cells remains an issue. Retinoic acid was used to differentiate the teratocarcinoma NT2 cell line into post-mitotic neurons (hNT cells) (Newman et al., 2005), and no signs of tumorigenicity were reported after a 2 year follow up in stroke patients (Kondziolka et al., 2005). In one transplanted stroke patient who died of a myocardial infarct 27 months after injection of the cell, autopsy revealed survival of the hNT cells with no deleterious effects of inflammation (despite only 2 months of immunosuppression) and no tumor formation (Nelson et al., 2002). ReNeuron took a different approach and created a conditionally immortalized cell line where c-myc is only active in the presence of tamoxifen (Stroemer et al., 2008); a successful Phase I clinical trial with these ReN001 cells will set precedence for such a strategy.

### (3) Blood, Bone Marrow, and Adipose Tissue-Delivered Progenitor Cells

The majority of stroke and progenitor cell transplantation studies employed non-neural cells: human bone marrow cells (HBMC), human umbilical cord blood cells (HUCBC), peripheral blood progenitor cells, and adipose tissue mesenchymal progenitor cells have all been reported to enhance recovery after stroke with intracerebral or intravascular delivery, and with acute (1 day), sub-acute (1 week), or chronic (1 month) delivery after stroke (Bliss et al., 2007; Guzman et al., 2008a; Hicks and Jolkkonen, 2009; Shen et al., 2007b). HBMC and HUCBC are composed of many cell types including hematopoietic and endothelial stem/progenitor cells (CD34++). MSCs (CD34−), and immature lymphocytes and monocytes (Erices et al., 2000; Newman et al., 2004; Nieda et al., 1997; Parr et al., 2007; Vendrame et al., 2004). It is not clear which cells are important to elicit recovery as enhanced function is reported with different cell populations (reviewed in (Bliss et al., 2007)).

These cells lack the ethical issues associated with embryonic- and fetal-derived cells. They are easily obtained offering the potential of autologous transplants, obviating the need for immunosuppression regimes. Even with xenogenic transplants, MSCs are thought to be hypoimmunogenic, as they do not initiate T cell priming or humoral antibody production (Li et al., 2002; Li et al., 2006b; Ryan et al., 2005). However, these cells show poor survival when injected, due either to lack of trophic support or through triggering the innate immune system. Such poor survival may be a disadvantage of these cells, although functional recovery is sustained out to one year (Shen et al., 2007a). Another advantage of these cells is that they are already in clinical use for malignant and non-malignant disorders (Horwitz et al., 1999; Koc et al., 1999; Wakitani et al., 2004), and have been tested in a stroke clinical trial (Bang et al., 2005). Two additional stroke clinical trials are also planned, one using autologous CD34+ bone marrow cells delivered into the middle cerebral artery (ClinicalTrials.gov Identifier: NCT00535197), the other using intraarterial delivery of autologous bone marrow mononuclear cells (ClinicalTrials.gov Identifier: NCT00473057).

### Other Cell Safety and Manufacturing Issues

Most cells transplanted in experimental stroke models are a heterogeneous population. As discussed above, HBMCs and HUCBCs contain multiple cell types, as do NPCs, which are composed of multipotent stem/progenitor cells in addition to cells already committed to a neuronal or glial fate. Dyskinesia is a major side effect in Parkinson's patients that have undergone tissue transplantation therapy. Recent work by Carlsson et al (Carlsson et al., 2009) revealed that the ratio of dopaminergic and serotoninergic grafted neurons play an important role in dyskinesia development, with an increasing ratio of serotonergic neurons being detrimental. Understanding the ‘active population’ will also be important for cell manufacturing as cell populations drift with time in culture and it will be necessary to monitor this to derive a clinically active product. Furthermore, cell karyotype must also be monitored as changes can occur with time in culture leading to aneuploidic cells.

### Potential Mechanisms of Transplanted Cell-Mediated Recovery

It is valuable to understand how cell transplantation affects the host brain. This knowledge will help elucidate the mechanism of action of the transplanted cells, improve their efficacy, and perhaps more importantly, it will also highlight potential side effects. As questions of mechanism are addressed it will be key to investigate both the spatial and temporal effects of the transplanted cells as ‘too much of a good thing can be bad’, as will be discussed. It is likely that transplanted cells will have multiple modes of action which will be dependent on cell type, timing and route of administration.

Integration into the Host Brain

An attraction of NPCs cells is their potential to replace lost circuitry; however evidence for this is limited. Transplanted NPCs in a rat model of global ischemia (Toda et al., 2001) and hNT neurons in a model of traumatic brain injury (Zhang et al., 2005) have been reported to express synaptic proteins. In the ischemic brain, electron microscopy studies revealed that human NPCs form synapses with host circuits (Ishibashi et al., 2004, Daadi et al., 2009a), and two groups demonstrated that hNPCs had electrophysiological properties characteristic of functional neurons (Buhnemann et al., 2006, Daadi et al., 2009a). However, only very few synapses are seen, and recovery occurred too early to be attributable to newly formed neuronal connections (Englund et al., 2002; Song et al., 2002). Moreover, recovery is also reported with non-neuronal cells (e.g. MSCs). Together, this implies that neuronal replacement is not a major contributor to cell-induced recovery.

Astrocytes play multiple roles in the brain including neuroprotection (Chen and Swanson, 2003; Panickar and Norenberg, 2005), regulation of synapse formation and activity (Allen and Banes, 2005), and involvement in the neurovascular unit which is important for blood brain barrier maintenance and coordinating blood supply with brain activity (Lok et al., 2007). Therefore, integration of astrocytes into the host brain will be as important as neuronal integration. White matter damage is another hallmark of stroke injury and replacement of lost oligodendrocytes to remyelinate axons would be beneficial. Remyelination by human NPCs was reported in spinal cord injury (Cummings et al., 2005), however, to date there are few reports of transplanted NPCs becoming oligodendrocytes in the ischemic brain (Daadi et al., 2008; Daadi et al., 2009a).

### Neuroprotection

Acute cell delivery, within the 48 h post-injury, often reduces lesion size and inhibits apoptosis in the penumbra, suggesting an important role for cell-induced neuroprotection in enhancing recovery (reviewed in (Bliss et al., 2007; Guzman et al., 2008a; Hicks and Jolkkonen, 2009)). A myriad of cells types elicit this effect, from NPCs, to bone marrow- and blood-derived cells. Common to all is the secretion of trophic factors such as vascular endothelial growth factor (VEGF), fibroblast growth factor (FGF), glial cell-derived neurotrophic factor (GDNF), and brain-derived neurotrophic factor (BDNF) that are likely to contribute to this neuroprotective mechanism (Kurozumi et al., 2005; Llado et al., 2004).

### Immunomodulation

Attenuation of stroke-induced inflammation is an emerging effect of transplanted cells. Intravenous injection of HUCBC (Vendrame et al., 2005) or human NPCs (Lee et al., 2008) reduced leukocyte numbers in the brain. Direct injection of human MSCs into the hippocampus after global ischemia downregulated many inflammatory and immune response genes and shifted the balance from a pro- to anti-inflammatory response (Ohtaki et al., 2008). These studies used acute cell delivery, and whether the results are a direct effect on the inflammatory response or a secondary effect attributable to a reduction in cell death is not clear. However, MSCs and NPCs can suppress T cell proliferation and modulate T cell induction in vitro possibly by releasing immunosuppressive cytokines and factors (Einstein et al., 2001; Nasef et al., 2007; Ryan et al., 2005; Tse et al., 2003; Yanez et al., 2006). Understanding the temporal effect of transplanted cells on inflammation will be critical as, like many therapeutic targets for stroke, inflammation has a dual role (Lo, 2008); during the acute phase, inflammation is detrimental mediating cell death, but in the recovery phase inflammation is beneficial for removal of cellular debris and neurovascular remodeling. Therefore, long-term attenuation of inflammation may not be advantageous.

### Enhancing Endogenous Repair Processes

There is mounting evidence that cell therapies can enhance many endogenous repair processes that occur after stroke.

(a) Vascular regeneration: Increased vascularization in the penumbra within a few days after stroke correlates with improved neurological recovery in patients (Krupinski et al., 1993; Senior, 2001) and offers a potential target for cell therapy. Transplanted cell-induced blood vessel formation has been reported with BMSCs, NPCs, HUCBCs and cells from human peripheral blood (Chen et al., 2003b: Horie et al., 2009b: Shen et al., 2006: Shyu et al., 2006: Taguchi et al., 2004). The ability of transplanted cells to increase levels of angiogenic factors (e.g. VEGF, FGF, GDNF, BDNF) and chemoattractant factors (e.g. SDF-1), either by direct secretion of the factor or by inducing host expression (Chen et al., 2003b), is likely to be important to stimulate proliferation of existing vascular endothelial cells (angiogenesis) and mobilization and homing of endogenous endothelial progenitors (vasculogenesis). Understanding the spatial and temporal effect of the transplanted cells on the vasculature is critical as uncontrolled vascularization can be detrimental as with retinopathies (Aiello, 2000). Recent data from our group finds that transplanted NPCs enhance vascularization in the penumbra but not in healthy tissue, and that the initial increase in vessels is followed by vascular regression, which mirrors the vessel dynamics in control animals (Horie et al., 2009b). This is important therapeutically as it demonstrates that NPCs affect the vasculature in a tightly controlled manner.

(b) Induction of host brain plasticity: An increase in endogenous brain structural plasticity and motor remapping after ischemia is postulated to underlie the spontaneous recovery seen after stroke (Carmichael, 2006; Carmichael, 2008; Dancause et al., 2005; Stroemer et al., 1995), and cell transplantation may enhance this. HUCBCs increased sprouting of nerve fibers from the contralateral to the ischemic hemisphere (Xiao et al., 2005), and our group has observed a similar phenomenon with fetal-derived NPCs (Horie et al., 2009a) and hES-derived NPCs (Daadi et al., 2009b). We have also observed that NPCs enhance dendritic branching in both the ipsi- and contralesional hemispheres (Horie et al., 2009a). Shen et al (Shen et al., 2006) reported increased synaptophysin expression after intravenous delivery of HBMCs, and we found that NPCs enhance synaptogenesis in vitro, which is partly mediated by thrombospondin secretion. While these data are cause for hope, a note of caution must be taken from Hofstetter et al (Hofstetter et al., 2005) who found that NPCs grafted in a model of spinal cord injury induced aberrant axonal sprouting which was detrimental, leading to allodynia-like forepaw hypersensitivity.

(c) Recruitment of endogenous progenitors: Endogenous neurogenesis increases after stroke (Arvidsson et al., 2002; Jin et al., 2001). The function of this is not understood but may signify a natural repair mechanism of the brain that could be enhanced by transplanted cells. There is precedence for this with cord blood- and bone marrow-derived cells (Chen et al., 2003a; Taguchi et al., 2004). Moreover, MSC-treated rats demonstrated elevated oligodendrocyte precursors, which increased in concert with enhanced white matter areas (Li et al., 2005; Li et al., 2006b; Shen et al., 2006). In addition to local effects on the damaged tissue, transplanted cells may recruit progenitors from other tissues. For example, they could mobilize endogenous endothelial progenitors into circulation to enhance vascularization.

In vivo Monitoring of Cell Therapy

Clinical studies will benefit from non-invasive methods to monitor the transplanted cells. Several imaging techniques like magnetic resonance imaging (MRI) (Guzman et al., 2001; Yano et al., 2005), bioluminescence imaging (BLi) (Love et al., 2007), positron emission tomography (PET) (Love et al., 2007), and in vivo fluorescence microscopy (Yano et al., 2005) have been used to track transplanted stem cells in vivo. Tagging the cells with nanoparticles (such as superparamagnetic iron oxide (SPIO)) allows them to be monitored by MRI (Weissleder et al., 1991). Several studies have demonstrated the feasibility to longitudinally track transplanted stem cells in experimental models of stroke (Guzman et all, 2007; Hoehn et al., 2002; Modo et al., 2004) and in a clinical trial for traumatic brain injury (Zhu et al., 2006). However, as released iron oxide particles from dead cells give the same MR signal, MRI cannot assess graft survival, and dilution of SPIOs when cells proliferate may significantly affect longitudinal studies (Guzman et al., 2007; Yano et al., 2005). BLI overcomes this issue, as it requires expression of the luciferase reporter gene that is transfected or transduced into the cells prior to transplantation. After administration of the substrate D-luciferin, cells can be tracked by quantification of photon emission. Since this modality depends on an active enzyme, it allows assessment of transplanted cell viability. However, current BLI methods provide only two-dimensional images with low spatial resolution, and downregulation of luciferase expression would give a false negative result. Transplantation of cells harboring a PET reporter gene (Yaghoubi and Gambhir, 2006) is another approach to track cells in vivo. The detection threshold of PET is 7 log orders more sensitive than MRI (Gambhir et al., 2000), but the disadvantage is that PET has a low spatial resolution and lack of anatomical information. In the future, multimodal imaging, combining MRI, BLI, and PET imaging techniques, will be used to combine the strengths and off-set the limitations of each technique (Ray et al., 2007); combined BLI and PET has been used in clinical trials involving patients with recurrent gliomas (Jacobs et al., 2001).

In addition to monitoring the transplanted cells, in vivo imaging are also be necessary to monitor the response of the brain to cell therapy. Functional imaging studies including PET (to monitor metabolic activity), perfusion studies (to monitor neovascularization and blood flow), functional MRI (to monitor cerebral plasticity), diffusion-tensor imaging (to evaluate fiber tract integrity), and MRI tracking of macrophages that have phagocytosed ultrasmall SPIO particles (to monitor brain inflammation) (Dousset et al., 1999; Jander et al., 2007; Rausch et al., 2001; Wiart et al., 2001), are useful surrogate clinical indicators of graft efficacy. Finally, cells, in vivo imaging may be useful to predict, on a patient-by-patient basis, when the brain microenvironment is optimal for cell transplantation. For example, MRI and PET techniques allow one to predict if a patient still has a penumbra (Baron, 2001; Schlaug et al., 1999). And, if enhancing vascularization is important for cell-mediated recovery it may be preferable to transplant when the angiogenic VEGF receptor is upregulated; we have recently demonstrated the feasibility of in vivo ⁶⁴Cu-DOTA-VEGF₁₂₁ PET imaging for investigating VEGFR expression kinetics (Cai et al., 2009).

Below is a tabularized summary of some previous studies relating to the use of stem cell trials in clinical studies for treating stroke.

TABLE 1
Clinical stem cell trials for stroke.

| Study | Cell Type | Source | n | Delivery | Stroke | Location | Stroke Age | Results |
|---|---|---|---|---|---|---|---|---|
| Kondziolka | hNT | Immortalized | 12 | IC (1x) | IS | Striatum | 6 months | Feasibility |
| et al., 2000 | | cell line | | | IS | | to 6 years | and safety |
| Kondziolka | hNT | Immortalized | 18 | IC (1x) | IS + HS | Striatum | 12 months | Feasibility |
| et al., proven | | cell line | | | IS + HS | | to 6 years | and safety |
| 2005 recovery | | | | | | | | Tendency |
| | | | | | | | | for clinical |
| Savitz et al., | LGE | xeno/swine | 5 | IC (1x) | IS | Striatum | 3 months | Terminated |
| due 2005 | | | | | IS | | to 10 years | by the FDA |
| | | | | | | | | to possible |
| | | | | | | | | side effects |
| Bang et al., | BMSC | autologous | 5 | IV (2x) | IS | Striatum + | 4-5 weeks | Feasibility |
| proven 2005 | | | | | | Cortex | 7-9 weeks | and safety |
| | | | | | | | | Clinical |
| | | | | | | | | recovery |

Abbreviations: LGE, lateral ganglionic eminence; RMSC, bone marrow-derived stem cells; IC, intracerebral; IV, intravenous; IS, ischemic stroke; HS, hemorrhagic stroke; FDA, Food and Drug Administration

### Co-Administration of Immunosuppressants

In accordance with another aspect of the present invention, co-administration of immunosuppressants may be used to inhibit the action of T cells, which would otherwise interfere with the therapy.

Generally, any immunosuppressant may be used if effective against T cells. For example, tacrolimus or cyclosporinA (CSA) are examples of such an immunosuppressant. Such an immunosuppressant may is be used in a conventional manner for a period of up to about 3-9 months post-transplantation, preferably for up to about 6 months post-transplantation.

More generally, immunosuppressants may be used as described as in U.S. Pat. No. 7,537,756, which is incorporated herein by reference in the entirety.

For example, immunosuppressants such as any cyclosporin, or FK-506 or even certain antibodies.

Further, as disclosed in U.S. Pat. No. 7,537,756, the immune response may be prevented or merely delayed or the intensity thereof diminished.

Other immunosuppressive agents which may be used include, for example, cyclophosphamide, prednisone, dexamethasone, methotrexate, azathioprine, mycophenolate, thalidomide, and systemic steroids.

Generally, the treatments described herein may be applied to any mammal, including human, mouse, rat, sheep, monkey, goat, rabbit, hamster, horse, cow or pig. Thus, the present invention has applicability to both research and clinical uses.

Having described the present invention, reference will now be made to certain examples which are provided solely for purposes of illustration and which are not intended to be limitative.

As noted above, recovery from neurological deficits will depend on restoration of axonal function. Many circuits remain intact after stroke, but are potentially impaired due to disruption of axonal function; this is particularly relevant to white matter tract injury. Axonal transport is critical for neuron function; transport of molecules and organelles between the cell body and the axon terminal is essential for neurotransmission. Furthermore, axonal transport is critical for the relay of molecules that signal axonal sprouting or degeneration, and synapse formation or retraction. Therefore, restoration of impaired axonal transport after stroke is likely key for both the proper functioning of existing axons and for plasticity changes such as axonal sprouting and synaptogenesis.

The factors involved in signaling axonal and dendritic changes after stroke have not been well understood but many molecules are known to promote neurite sprouting either in culture or in vivo. Such molecules include neurotrophic factors like vascular endothelial growth factor (VEGF) and basic fibroblast growth factor, extracellular matrix molecules like the thrombospondins (TSPs) and SPARC (Secreted Protein Acidic and Rich in Cystein and factors important for neurite growth and guidance during development like Slit. Neural progenitor cells express many such factors known to influence neurite plasticity and thus have the potential to enhance structural plasticity after stroke. Therefore, in the present study, we addressed the effects of human neural progenitor cell (hNPC) transplantation on dendritic and axonal plasticity and axonal transport in rats after stroke, and correlated this with hNPC-induced recovery. Furthermore, we investigated the potential of several hNPC-secreted factors to induce such plasticity and transport changes in vitro.

### EXAMPLE 1

Transplanted hNPCs Survive in the Ischemic Host Brain and Enhance Functional Recovery hNPCs transplanted into the rat cortex 7 days post-stroke exhibited robust survival and migration towards the lesion at 5 weeks post-transplantation (FIG. 3A). The majority of the hNPCs stained with nestin (78.2±6.0%) implying that most cells remained in an immature state. A small proportion of cells expressed the astrocyte marker glial fibrillary acidic protein (GFAP; 17.2±7.9) or the immature neuronal marker β-Tubulin (TuJ1; 4.6±2.2%) (FIG. 1B, C). No co-localization was observed with the oligodendroglial markers NG2 and Olig2. Lesion size was not significantly different between hNPC- and vehicle-treated rats at 5 weeks post-transplant (21.2±3.7 mm³ and 15.3±1.9 mm³, respectively). However, hNPC-grafted rats showed significantly enhanced functional recovery in 3 out of 4 behavior tests compared to vehicle controls (MANOVA P<0.05) which started between 3 to 4 weeks post-transplantation.
hNPC Transplantation Promotes Dendritic Plasticity

Golgi-stained dendrites from layer V cortical pyramidal neurons (FIG. 5C) were analyzed between the lesion and the graft (and the corresponding ROI in the contralateral cortex). hNPC treatment enhanced dendritic branching and total dendritic length at 2 weeks post-transplantation in both hemispheres compared to vehicle controls (FIG. 5A, B). Of note, vehicle-injection appeared detrimental to dendritic plasticity in the injected (ipsilesional) hemisphere compared to non-treated stroke animals at this time point (FIG. 5A, B). hNPC-induced dendritic changes in the contralesional hemisphere abated by 4 weeks post-transplant. In contrast, the effects in the ipsilesional hemisphere were sustained suggesting that proximity to the hNPCs is important to maintain enhanced dendritic plasticity. Changes in dendritic branching were more significant in basilar branches compared to apical branches (FIG. 5D-F), and the greatest plasticity was observed in the middle order branches (branch order 3-7) rather than in branches emanating from the soma or at the dendrite extremities (FIG. 5E, F).

### EXAMPLE 2

hNPC Transplantation Promotes Axonal Rewiring after Stroke

The anterograde axonal tracer biotinylated dextran amine (BDA) injected into the contralesional cortex was used to visualize axons. hNPC-grafted rats, compared to vehicle-treated rats, appeared to increase axonal sprouting from the contralesional cortex to the ipsilesional hemisphere (FIG. 7A). This was first evident at 3 weeks post-transplantation with increased BDA-labeled fiber density in the corpus callosum and ipsilesional striatum of hNPC-treated animals (FIG. 7B), and more pronounced at 5 weeks post-transplantation with increased cortico-cortico, cortico-striatal, and cortico-thalamic sprouting as evidenced by significantly increased BDA-labeled fiber density in the relevant regions of interest as indicated in FIG. 7B,C. Furthermore, hNPC-grafted rats showed enhanced corticospinal tract projections at 5 weeks post-transplantation with significantly increased BDA-labeled fiber density in the contralesional internal capsule and both the contra- and ipsilesional dorsal funiculus of the cervical spinal cord (FIG. 7D). To further substantiate hNPC-induced plasticity, we found that hNPC treatment significantly enhanced expression of the axonal growth cone protein GAP-43 in the corpus callosum and cortex of both hemispheres, with the largest increase in the ipsilesional cortex (FIG. 8). BDA labeling in the corpus callosum at 5 weeks post-transplantation in hNPC-treated animals positively correlated with functional recovery in the whisker-paw (p=0.005) and cylinder tests (p=0.04). Recovery in the whisker-paw test also positively correlated with the BDA signal in the injured CST in hNPC-treated rats (p=0.023). These data suggest that hNPC-induced axonal changes are important for hNPC-enhanced recovery.

### EXAMPLE 3

### Identification of hNPC-Secreted Factors that Modulate Dendritic and Axonal Plasticity

In a non-contact co-culture assay (FIG. 9) hNPCs significantly increased dendritic branching (FIG. 13A, B), total dendritic length (FIG. 13A, C), and axonal length (FIG. 13A, D) of co-cultured cortical neurons, thus mimicking in vitro the hNPC-mediated effects on dendritic and axonal plasticity observed in vivo. To identify potential hNPC-secreted plasticity mediators, immunodepletion studies were done for specific candidate molecules known to be involved in neuronal plasticity and expressed in our hNPCs by microarray analysis (Wright et al., 2003). Neutralization of thrombospondin 1 (TSP1) or TSP2 significantly reduced hNPC-induced dendritic branching and length, while neutralization of human VEGF (hVEGF) using Avastin, or of Slit only affected total dendritic length with no effect on dendritic branching (FIG. 13B, C). hNPC-mediated axonal outgrowth was significantly reduced by neutralization of all the aforementioned factors (FIG. 13D); neutralization of TSP1 and TSP2 also inhibited axonal growth in vehicle-treated cortical neurons. Depletion of SPARC had no effect on either dendritic or axonal morphology (FIG. 13B-D). The effects of the neutralizing antibodies were specific as isotype control antibodies had no effect (P>0.05).

### EXAMPLE 4

Transplanted hNPCs Reduce Impairment of Axonal Transport Post-Ischemia

Disruption of anterograde axonal transport was quantitatively assessed by measuring amyloid precursor protein (APP) accumulation in axons (labeled with the neurofilament marker SMI312). There was significant accumulation of APP in axons in the corpus callosum after stroke (FIG. 14A, B), consistent with previous reports of stroke impaired axonal transport (Valeriani et al., 2000, Wakita et al., 2002). hNPC-grafted animals had significantly fewer APP-positive axons at 3 weeks compared to vehicle controls (FIG. 14B), and this difference was further enhanced by 5 weeks suggesting that hNPCs enhance recovery of axonal transport. Furthermore, at 5 weeks hNPC animals had more axons in the corpus callosum as determined by the number of SMI312 puncta (FIG. 14C). Together, these data imply that hNPC-treated animals have more functioning axons (i.e., axons without impaired transport) after stroke than vehicle controls. Moreover, APP accumulation in the corpus callosum showed an overall negative correlation with recovery in the whisker-paw (p=0.004) and cylinder test (p=0.02) when combining data from both vehicle and hNPC groups, suggesting that reducing APP and improving axonal transport is important for recovery.

### EXAMPLE 5

Effects of hNPCs on Axonal Transport are Mediated by VEGF

The effect of hNPCs and hNPC-secreted factors on axonal transport was determined in vitro using a microfluidic platform with one chamber seeded with primary cortical neurons and the other with or without hNPCs (FIG. 14D). Anterograde transport of dextran-labeled vesicles in the axons of the cortical neurons was measured in the axonal compartment (FIG. 14E). Co-culture with hNPCs significantly increased the mean velocity of anterogradely transported vesicles (FIG. 14F, FIG. 15). Neutralization of VEGF, but not TSP1, TSP2, SPARC, or Slit inhibited the hNPC-mediated effect (FIG. 14F). Isotype control antibodies had no effect, and neutralization of these factors had no effect in vehicle-treated wells.

Thus, the present inventors have demonstrated that hNPC transplantation at 1 week after stroke significantly improved functional recovery in several behavior tests and that this recovery correlated with hNPC-enhanced changes in dendritic and axonal structural plasticity. hNPCs not only elicited these plasticity changes in the injured hemisphere but also helped recruit the uninjured hemisphere. Moreover, the present inventors have demonstrated for the first time that stem cell transplantation enhanced recovery of stroke-impaired axonal transport. This is significant given the vital role of axonal transport for neuron function, survival, and plasticity. Using co-culture assays, we could mimic these stem cell-induced effects on plasticity and transport in vitro, and through neutralization studies we identified 4 secreted factors that mediate these hNPC-induced effects. These data suggest that transplanted hNPCs enhance post-stroke recovery by secretion of factors that enhance endogenous brain repair mechanisms induced after stroke injury.

### Stem Cells Enhance the Innate Repair Capacity of the Brain

There is compelling evidence from both patient and animal data that the brain undergoes reorganization and rewiring of surviving circuits after ischemia and this is postulated to underlie the spontaneous recovery observed after stroke (Benowitz and Carmichael, 2010, Dancause, 2006, Murphy and Corbett, 2009). Such restorative neuronal plasticity changes include an increase in afferent and efferent connections in both ipsi- and contralesional brain regions, resulting in part from changes in dendritic and axonal sprouting of surviving neurons.

Chronic changes in dendritic structural plasticity after stroke have been reported with increased contralesional layer V dendritic branching peaking at 18 days post-stroke (Jones and Schallert, 1992), while ipsilesional layer III branching was decreased (compared to uninjured animals) at 9 weeks post-stroke (Gonzalez and Kolb, 2003). Our data show for the first time that at 3 weeks post-stroke, hNPCs enhance dendritic length and arborization in layer V cortical neurons in both the ipsi- and contra-lesional cortex (despite the latter being remote from the site of transplantation), and this coincided with the onset of hNPC-induced functional recovery. Enhanced contralesional dendritic plasticity after stroke is thought to be due to compensatory increased use of the unimpaired limb (Jones and Schallert, 1994). However, this is not apparent in the hNPC-treated animals; despite enhanced contralesional plasticity they exhibit less relative use of the unimpaired limb than vehicle controls. Thus, the significance of hNPC-enhanced contralesional dendritic plasticity, and its importance for early recovery, remains to be determined.

hNPC-induced dendritic changes in the contralesional cortex were transient returning to baseline by 5 weeks post-stroke, thus indicating that hNPCs could not prevent the contralesional branch regression previously reported (Jones and Schallert, 1992), and that contralesional dendritic changes are not necessary for continued recovery at later time points. In contrast, ipsilesional changes (i.e., in the hNPC-grafted hemisphere) were maintained at 1 month post-transplantation with no sign of abating, suggesting that local effects of the hNPCs are necessary to maintain dendritic changes. The biggest change in dendritic structural plasticity was observed in basilar dendrites where the majority of synaptic inputs are found (Larkman, 1991). This could imply hNPCs' ability to facilitate change is influenced by the amount of activity within a dendritic branch. The pattern of early dendritic changes in the contralesional cortex followed by a switch to more dominant changes in the ipsilesional cortex at later times is reminiscent of brain remapping results in patients and animals. These remapping studies show that stimulation of the injured limb early after stroke recruits the contralesional cortex and this switches back to the ipsilesional cortex at later time points (Benowitz and Carmichael, 2010, Dancause, 2006).

Axonal sprouting occurs after stroke with new projections thought to target areas denervated by the stroke injury (Benowitz and Carmichael, 2010). In rodent and primate models of ischemic cortical injury, such sprouting has been observed locally around the infarct area (Carmichael et al., 2001, Conner et al., 2005, Dancause, 2006), while interhemispheric axonal outgrowth from the intact cortex to the injured hemisphere has also been observed (Carmichael, 2008). Our axonal tracer (BDA) data indicate that hNPC transplantation promotes this interhemispheric cortical sprouting in cortico-cortical, cortico-striatal, and cortico-thalamic pathways, corroborating previous reports using human umbilical cord blood cells (Xiao et al., 2005) and human embryonic-derived neural stem cells (Daadi et al., 2010). Consistent with this, hNPCs enhanced expression of the axonal growth cone protein GAP43 in the same regions. However, GAP43 is not purely a marker of regenerating axons as it is also expressed on non-neuronal cells such as astrocytes and oligodendrocytes (Carmichael, 2008); the importance of this for regeneration is not understood.

hNPC transplantation also enhanced stroke-induced remodeling of cortical-spinal tract (CST) axons originating from the contralesional cortex (i.e., intact CST). The fact that the number of BDA-labeled fibers not only increased in the ipsilateral dorsal funiculus (intact CST) but also in the contralateral dorsal funiculus (injured CST) implies that hNPCs enhance collateral (cross midline) sprouting of the intact CST into denervated regions of the spinal cord (Chen et al., 2002, Liu et al., 2008). Bone marrow stromal cell (MSC) treatment enhanced this collateral sprouting (Liu et al., 2008), but unlike hNPC treatment, MSCs had no effect on fiber density in the intact CST (Liu et al., 2008). The hNPC-induced changes in both CST and trans-callosal axonal sprouting statistically correlated with hNPC-enhanced functional recovery; a similar correlation was found between recovery and MSC-induced CST sprouting (Liu et al., 2008). Together, these data indicate that hNPC-induced axonal plasticity is an important mechanism for hNPC-induced recovery.

A key provision of the present invention relates to the ability of neuropotentiating cells, such as hNPCs, to enhance recovery of axonal transport after stroke. This has major implications for recovery of white matter injury. Axonal transport is disrupted after ischemia (Kataoka et al., 1989, Valeriani et al., 2000, Wakita et al., 2002) and is indicative of axonal damage. APP is commonly used as a marker of impaired transport and axonal degeneration (Stone et al., 2000, Valeriani et al., 2000) as it is constitutively expressed in neurons and normally subject to fast axonal transport (Koo et al., 1990). However, under pathological conditions APP accumulates in axons, which is both a cause and consequence of disrupted transport. Our results confirm that stroke induces APP accumulation in corpus callosum, and it remains elevated at 6 weeks post-stroke, implying extended perturbation of axonal transport. hNPC transplantation reduced APP accumulation and accelerated the decrease of APP over time, which strongly suggests that hNPCs enhance recovery of fast axonal transport after stroke. Furthermore, by direct measurement of vesicle transport in cultured cortical neurons, we confirmed that hNPCs can enhance axonal transport. This is a significant finding as axonal transport is fundamental to neuron function, not only for proper functioning and survival of existing axons but also for plasticity changes such as axonal sprouting and synaptogenesis. Therefore, hNPC-induced restoration of impaired axonal transport after stroke may not only enhance the function of existing fiber tracts but may also be a key upstream event of hNPC-induced structural plasticity.

Identification of Putative Secreted Factors Involved in hNPC-Mediated Plasticity

It is postulated that transplanted stem cells elicit their effects through secretion of relevant factors. Using in vitro co-culture systems in which hNPCs mimicked the effects observed in vivo, we identified secreted molecules putatively involved in hNPC-induced axonal transport and neurite sprouting. We found that TSP1 & 2, VEGF, Slit, but not SPARC, were important for hNPC-induced dendritic and axonal outgrowth. In contrast, only TSP1 & 2 were important for hNPC-induced dendritic branching, consistent with the idea that neurite outgrowth and branching are activated by different pathways (Carmichael, 2008, Poulain and Sobel, 2010). Only VEGF was important for hNPC-enhanced axonal transport. VEGF has previously been reported to be transported by axons.

Because most antibodies used in these experiments may bind both human and rodent forms of the protein, further studies are required to determine whether direct secretion of these factors by the hNPCs or hNPC-induced secretion of these factors by the primary rat cultures is important for the observed effects. However, neutralization of VEGF was achieved using Avastin, which binds human but not rodent VEGF (Ferrara et al., 2004), strongly implying that direct secretion of VEGF by hNPCs plays a significant role.

In summary, we have demonstrated that transplanted hNPCs enhance axonal transport, dendritic branching and axonal sprouting of host neurons after stroke, and that these plasticity changes correlated with hNPC-induced recovery. Furthermore, we identified several secreted factors that mediated these same changes in vitro. Therefore, we assert that transplanted hNPCs, for example, aid in recovery through secretion of factors that enhance the natural brain remapping that occurs following stroke. Such a mechanism of action offers a large therapeutic window for post-stroke stem cell intervention.

As noted above, however, the present inventions also explicitly contemplates use of all types of neuropotentiating cells, particularly those mentioned above in the treatment of brain injury and spinal injury. Implicitly, the present invention also provides a method of promoting recovery after brain and spinal injury or surgery. Also, the present invention also provides a method of measuring axonal properties in mammals, particularly humans.

In each of the methods noted above, the steps involved involve: a) locating the lesion or lesions to be treated by taking an imaging scan, such as CT scan, of the patient's head; b) introducing an amount of neuropotentiating cells to the patient effective to enhance axonal properties in the patient's brain; and c) monitoring the enhancement by various techniques, such as fMRI or DTI, as mentioned above.

### Materials and Methods

Human NPC Culture Human NPCs, initially isolated from fetal cortical brain tissue at 13.5 wks gestation (M031 clone) were cultured as neurospheres in Stemline™ Neural Stem Cell Expansion Medium (Sigma) supplemented with 20 ng/ml epidermal growth factor (Sigma) and 10 ng/ml leukemia inhibitory factor (Chemicon) as previously described (Svendsen et al., 1998). For transplantation and in vitro experiments, NPCs (passage 16-25) were dissociated to a single cell suspension by incubation at 37° C. with Accutase (10 min; Sigma), trypsin inhibitor (5 min; Sigma), and DNase (10 min; Sigma), followed by gentle trituration.

### Stroke Surgery and Cell Transplantation

Animal procedures were approved by Stanford University's Administrative Panel on Laboratory Animal Care. T cell-deficient adult male Nude rats (Cr:NIH-RNU 230±30 g; NCI-Frederick Cancer Research) were subjected to permanent dMCAo with 0.5 h bilateral CCA occlusion as described (Kelly et al., 2004) under isoflorane anesthesia. 1 mg/ml Ampicillin was administered orally 3 d before stroke surgery to 7 d post-transplantation surgery. Seven days post-dMCAo 3, 1.0 μl deposits of hNPCs (1×10⁵ cells/μl), or vehicle (0.9% saline), were injected into the ipsilesional cortex as described (Kelly et al., 2004): (i) anterior-posterior (A-P), +1.0; medial-lateral (M-L), −1.2; dorsal-ventral (D-V), −2.7; (ii) A-P, −0.26; M-L, −1.2; D-V, −2.6; (iii) A-P, −1.8; M-L, −1.0; D-V, −1.8.

### Behavioral Testing

Behavior was tested pre-operatively for baseline performance and repeated weekly for 6 wks post-dMCAo (blinded). (a) Vibrissae-elicited forelimb placing test 10 trials of the vibrissae-evoked forelimb placing test was done on each side as described previously (Schallert et al., 2000). (b) Cylinder test (Schallert et al., 2000): Rats were placed in a plastic cylinder and the number of times they reared and touched the cylinder in a weight-bearing fashion with the left (L), right (R), or both forelimbs was counted for 20 hits. The ratio R:L ratio was calculated. (c) Elevated body swing test (Borlongan and Sanberg, 1995): Rats were lifted by the proximal tail 3 cm above the table, the direction of swing in order to reach upward was recorded for 10 trials. (d) Postural reflex test (Bederson et al., 1986): animals were suspended by the proximal tail, 1 m above ground, and slowly lowered. Symmetric touch-down with both forelimbs scored 0 points and testing ended. Animals with abnormal postures (rotating body, flexing of limbs) were placed on the table and gently pushed side-to-side. Rats resistant to pushing scored 1 point, animals unable to resist scored 2 points.

### Tissue Processing and Immunostaining

Rats were perfused as previously described (Kelly et al., 2004), brains and cervical spinal cords removed, and 30□ brains and cervical spinal Immunostaining was performed conventionally (Kelly et al., 2004) using primary and secondary antibodies as described in Supplementary Methods, followed by confocal microscopy analysis. To assess the hNPC differentiation profile, Z-stacks were acquired and 100 or more HuNu-positive cells were scored for each marker (Nestin, TuJ1, GFAP, NG2, Olig2). GAP43 pixel intensities were analyzed in 6 slices encompassing the entire lesion using ImageJ.

Analysis of Axonal Transport in vivo

Immunohistochemistry for axonal APP was performed as previously described (Stone et al., 2000); Supplementary Methods) using rabbit anti-APP (c-terminus; 1:2000; Invitrogen) with a mouse anti-SMI312 antibody (1:1000; Sternberger Monoclonals). Confocal images were acquired from 6 sections/animal (approximately 500 μm apart) in 2 regions of interest (ROI)/section: the ipsilesional and contralesional genu of the corpus callosum The total number of SMI312-positive fibers/ROI and the ratio of APP/SMI312-co-localizing axons were analyzed using the “Puncta Analyzer” plugin for NIH ImageJ software, as described previously (Liauw et al., 2008); Supplementary Methods, and averaged over the 6 sections.

### Analysis of Infarct Size

Infarct size was determined on 5 cresyl violet stained sections taken at 480 μm intervals between the levels +1.2 to −1.6 mm from bregma and expressed as percentage of the contralateral, non-ischemic hemisphere as described previously (Kelly et al., 2004).

### Dendritic Analysis

Rats were anesthetized with isoflurane, the brains removed and stained using a Rapid GolgiStain™ Kit (FD NeuroTechnologies), and 150 μm coronal sections cut. Layer V pyramidal neurons were analyzed (blinded) in the region between the lesion and the hNPC graft, and the equivalent location in the contralesional cortex, i.e., between Bregma and Bregma −1.2 mm, between the dorsal peak of the corpus callosum and up to 4 mm from the midline. To be included, neurons had to be: well impregnated, in full view with no overlapping blood vessels or astrocytes, appear intact and in the plane of section. The length of each dendritic branch was determined using the measuring tool on the StereoInvestigator software (MicroBrightField) and following the dendrite through the Z axis; the number of branches was tabulated at the same time. Branch order was analyzed as shown in FIG. 2 (Coleman and Riesen, 1968).

### Axonal Tracing Studies

At 2 or 4 wks post-transplantation, 0.2-μl of the anterograde axonal tracer biotinylated dextran amine (BDA, MW 10,000, 0.1 μg/μl; Molecular Probes) was injected at 0.1 μl/min into the contralesional layer V cortex at: (i) A-P, −1.0; M-L, −1.3; D-V, −1.8; (ii) A-P, −1.0; M-L, −1.8; D-V, −1.8; (iii) A-P, −0.5; M-L, −1.3; D-V, −1.8; (iv) A-P, −0.5; M-L, −1.8; D-V, −1.8. The needle was left in situ 5 min then slowly removed. 1 wk later brains and cervical spinal cord were processed for immunohistochemistry: after 30 min permeabilization with digitonin (100 μg/ml; Calbiochem), BDA was detected using streptavidin-conjugated antibody (Alexa Fluor 546, 1:200; Molecular Probes). Confocal images were acquired from different ROIs in 6 brain slices encompassing the entire ischemic area. BDA-positive fiber densities were calculated using ImageJ software and the values normalized to the number of BDA-positive cell bodies.

In vitro Dendritic and Axonal Outgrowth Assays

Primary cortical cultures from Sprague-Dawley rat E14 embryos (Charles River Laboratories, MA) were prepared as described (Andres et al., 2005); Supplementary Methods. After 3 d in vitro (DIV) hNPCs (180,000 cells) or vehicle were added to the primary cultures in cell culture inserts (Falcon 353095). For immunodepletion studies, neutralizing antibodies (Supplementary Methods) were added to the medium in the wells and inserts at 5 μg/ml from DIV3-7. At DIV7 the cortical cultures were fixed and axons labelled with anti-SMI312 (1:1000) and dendrites with anti-microtubule-associated protein 2 (MAP2) (1:400; BD Pharmingen, CA), followed by secondary antibodies (Alexa Fluor 488 or 546 conjugated goat anti-mouse, 1:250; Molecular Probes). Fluorescent images of randomly selected fields were acquired and neurite outgrowth analyzed using an automatic neurite tracing software (HCA Vision V1.6.5, CSIRO, Australia). Per group, 42-458 individual cells, from 3-6 independent experiments were analyzed.

### Microfluidics Axonal Transport Assay

E14 rat embryonic primary cortical cultures were grown in the ‘cell body’ chamber of a microfluidics device (Taylor et al., 2005); see Supplementary Methods. At DIV3, 120,000 hNPCs or vehicle were added to the distal chamber. At DIV 5, when axons of the primary cultures had extended through the microgrooves, Alexa Fluor 488-conjugated dextran (0.1 mg/ml; 10,000 MW, anionic, Nr. D-22910, Invitrogen) was added to the cell body chamber, and anterograde transport of dextran-labeled vesicles was analyzed as reported (Kunzi et al., 2002). In brief, 2 h after dextran labeling, time-lapse fluorescent images were acquired every 12.5 seconds. Transport velocities of individual anterogradely-transported vesicles were calculated if they were identifiable for at least 6 frames in a series. Vesicles with velocities <0.1 μm/sec were discarded to exclude movements caused by Brownian motion (Kunzi et al., 2002). Immunodepletion studies were carried out as described above with antibodies added to both the cell body and distal compartments. A total of 140-840 vesicles were analyzed/group, from 4 independent experiments.

### Statistical Evaluation

Statistical analysis utilized GraphPad Instat (GraphPad Software) and JMP 8.0.1 (SAS Institute). In vivo dendritic data analysis consisted of repeated measures analysis of variance using PROC GLM, a procedure in SAS, to compare treatment effects after adjusting for subject effects. ANOVA assumptions were examined with residual plots. For all other data experimental groups were compared by analysis of variance (ANOVA), followed by nonparametric post-hoc Mann-Whitney test, or Student's t-test. BDA or APP values (from the genu of the contralateral corpus callsoum) were tested for correlation with functional recovery (defined as change in behavior from wk 1 to wk 6 post-stroke) using the Pearson test. Differences were considered statistically significant at P<0.05. Values are presented as mean±SEM.

### Funding

This work was supported by National Institutes of Health National Institute of Neurological Disorders and Stroke [NS058784, NS27292, NS37520 to G.K.S.]; the National Institute of Health [P01 NS057778 to C.N.S.]; the William Randolph Hearst Foundation, Bernard and Ronni Lacroute, Russell and Elizabeth Siegelman, John and Dodie Rosenkrans, and the Edward E. Hills Fund (to G.K.S.); and Swiss National Science Foundation Grants [PBBEB-117034 and PASMP3-123221/1 to R.H.A.]; and the Evelyn L. Neizer Fund (to R.H.A).

P<0.01. n=12 per group, except cylinder test n=6. Tx=transplantation. Scale: A: 50 μm, C: 10 μm.

### Supplementary Materials and Methods

### Tissue Processing and Immunostaining

Primary antibodies were incubated overnight at 4° C.: mouse anti-HuNu (1:500, Chemicon); rabbit anti-Nestin (1:500, Abcam); chicken anti-TuJ1 (1:1000, Chemicon); guinea pig anti-GFAP (1:500, Advanced Immunochemical); rabbit anti-NG2 (1:500, Chemicon); rabbit anti-Olig2 (1:200, Abcam); rabbit anti-GAP-43 (1:2000, Chemicon). Secondary antibodies were incubated 1 h, room temperature (1:250 Alexa Fluor 556 or 488, Molecular Probes), with DAPI (1 μg/ml, AnaSpec).

Analysis of Axonal Transport in vivo

Immunohistochemistry for APP was performed as previously described (Stone et al., 2000). After thorough rinsing in TBS, antigen retrieval was performed by incubation of the slices in 10 mM sodium citrate (pH 6.5; Sigma) at 60° C. with gentle rocking for 20 min. Sections were rinsed and incubated with H₂O₂:Methanol:TBS (1:3:6, v:v:v) for 15 min at RT on a rocker, blocked in 5% normal horse serum (Vector) and 0.1% Triton X-100 (Sigma), and incubated with a rabbit polyclonal antibody to the c-terminus of APP (1:2000; Invitrogen) and a mouse monoclonal anti-SMI312 antibody (1:1000; Sternberger Monoclonals) for 24 h at 4° C. Sections from naïve rats and from double human APP mutant mice (T41B strain) served as negative and positive controls, respectively. Negative controls displayed only very low background fluorescence signal, while strong APP immunoreactivity was abundant in axons and cell bodies in sections from T41B mice.

For quantitative analysis of APP-immunoreactive axons, 6 sequential sections approximately 500 μm apart were selected from each animal. Confocal images were acquired in 4 regions of interests (ROIs) per slide in the ipsilesional and contralesional sides of the corpus callosum and cortex and APP- and SMI312 positive fibers were measured and co-localized using the “Puncta Analyzer” plugin for NIH ImageJ software, as described previously (Liauw et al., 2008). Gain thresholds and amplitude offsets were kept constant throughout the experiments. To exclude unspecific background signal, a band pass filter excluding structures with a diameter smaller than 0.5 μm or larger than 4.5 μm was applied. The total number of SMI312-positive fibers per area was determined and the ratio of APP/SMI312-co-localizing and SMI312-immunoreactive axons was calculated.

In vitro Dendritic and Axonal Outgrowth Assays

Primary cortical neural cells were isolated from Sprague-Dawley rat E14 embryos (Charles River Laboratories) as previously described (Andres et al., 2005). Cells were plated on poly-l-lysine (Sigma) coated coverslips (Nunc, Rochester) at 30 viable per mm², and grown in 0.5 ml of Neurobasal A medium (Invitrogen,) with 0.5 mM L-glutamine (Invitrogen) and 627 (Invitrogen). Antibiotics/antimycotics (No. 061-05240 D; Gibco) were present during the first 3 d in vitro (DIV). On DIV3, hNPCs (180,000 cells/insert) or vehicle were added to the primary cultures in cell culture inserts (pore size 0.4 μm, Falcon 353095, Becton Dickinson, NJ) with 300 DI medium. For immunodepletion studies, neutralizing antibodies were added to the medium in the wells and inserts at 5 pg/ml from DIV3-7: TSP1 and TSP2 (Santa Cruz Biotechnology), VEGF (Calbiochem) human VEGF (Avastin, Roche), human SPARC (R&D Systems), and Slit (recombinant rat ROBO1/Fc chimera, R&D Systems). Species-matched isotype control antibodies (BD Pharmingen) were also tested. Medium was changed every other day and antibodies re-added with each medium change. Cultures were fixed with 4% PFA at DIV7 and axons labeled with anti-SMI312 (1:1000; Sternberger Monoclonals, MD) and dendrites with anti-microtubule-associated protein 2 (MAP2) (1:400; BD Pharmingen, CA), overnight at 4° C., followed by secondary antibodies (Alexa 488 or 546 conjugated goat anti-mouse, 1:250; Molecular Probes) and DAPI. For quantitative analysis of neurite outgrowth, digital images of randomly selected fields from SMI312- or MAP2-immunostained cultures were acquired and analyzed using an automatic high-throughput neurite tracing software (HCA Vision V1.6.5, CSIRO Mathematical and Information Sciences, North Ryde, Australia). The total length of all dendrites per neuron, the number of dendritic branching points, and the average axonal length of each cell were calculated.

### Microfluidics Axonal Transport Assay

For in vitro analysis of anterograde axonal transport, a microfluidic culture platform (Taylor et al., 2005) and live imaging methods (Kunzi et al., 2002) were used as previously described, with some modifications. Chambers were fabricated in polydimethylsiloxane (PDMS) using soft lithography and replica molding. The microfluidic compartmented chamber was then assembled by placing the PDMS device onto a poly-L-lysine coated glass plate. The cell body chamber is separated from the distal chamber by microgrooves ranging from 100 μm to 450 μm in length, 5 μm wide and 3 μm high. The fluidic isolation is maintained by the array of microgrooves with higher hydrostatic pressure in the cell body chamber, in order to minimize tracer diffusion from the proximal chamber during live transport assay. E14 rat embryonic cortical neural cells were isolated as described above. 2000 cells were then seeded into the cell body chamber containing DMEM plus 10% normal horse serum for the first 12 h. Medium was then replaced by phenol red-free Neurobasal A (Invitrogen) supplemented with 0.5 mM L-glutamine (Invitrogen) and B27 (Invitrogen) and cells were cultured at 37° C. in a 5% CO₂/air humified atmosphere. At DIV3, 120'000 viable dissociated hNPCs or vehicle were added into the distal compartment. Axonal growth was monitored and reached the distal chamber through microgrooves around DIV 5. Alexa Fluor 488-conjugated dextran (10,000 MW, anionic, Nr. D-22910, Invitrogen) was added to the cell body compartment 2 h before imaging, at a concentration of 0.1 mg/ml, and anterogradely-transport of dextran-labeled vesicles was analyzed according to established protocols (Kunzi et al., 2002). In brief, live time-lapse images were acquired every 12.5 seconds using the confocal fluorescent microscope at 0.05% laser power in a 5% CO₂/air humified atmosphere after addition of 0.3 U/ml OxyFluor™ (Oxyrase Inc.) to the medium. Transport velocities of individual anterogradely transported vesicles were calculated if they were identifiable for at least 6 frames in a series. Vesicles with velocities <0.1 μm/sec were disregarded to exclude movements caused by Brownian motion (Kunzi et al., 2002 Kunzi et al., 2002). For investigating the effect of specific factors, immunodepletion studies were carried out for TSP1/2, VEGF, SPARC, and Slit as described above, with blocking antibodies or isotype controls added.

Having described the present invention, it will now be apparent that many changes and modifications may be made to the above-described embodiments without departing from the spirit and the scope of the present invention.

What is claimed is:

1. A method of assessing efficacy of transplanted neuropotentiating cells in promoting recovery from brain or spinal surgery, which comprises after conducting brain or spinal surgery on a mammal, and after transplanting said neuropotentiating cells into an affected area of the brain or spine respectively, measuring the affected area to determine:
a) dendritic branching,
b) total dendritic length,
c) branch order,
d) axonal sprouting, and/or
e) axonal transport.

2. The method of claim 1, wherein said mammal is a human.

3. The method of claim 1, wherein said transplanting is effected in at least the ipsilesional cortex.

4. The method of claim 1, wherein recovery after stroke is promoted.

5. The method of claim 1, wherein the neuropotentiating cells are MSCs, hNPCs, induced neurons, blood-, bone marrow- or adipose tissue derived cells or olfactory ensheathing cells.

6. A method of treating brain or spinal injury to a mammal, which comprises transplanting neuropotentiating cells to an affected area of said brain or spine of said mammal.

7. The method of claim 6, for treating a brain injury, wherein the hNPCs are transplanted to at least the ipsilesional cortex.

8. The method of claim 6, wherein the mammal is a human.

9. The method of claim 6, which promotes dendritic plasticity in said mammal.

10. The method of claim 6, which promotes axonal plasticity in said mammal.

11. The method of claim 6, which promotes axonal rewiring in said mammal.

12. The method of claim 6, wherein the neuropotentiating cells are introduced at multiple loci in or, at least, adjacent to the affected area, thereby encompassing the area.

13. A method of reducing impaired axonal transport in a post-ischemic mammal, which comprises transplanting neuropotentiating cells to an affected area of a brain or spine of said mammal, thereby reducing said impaired axonal transport.

13. A method of simulating in vivo effects of neuropotentiating cells in an in vitro cell culture assay, which comprises:
a) co-culturing neuropotentiating cells with animal model cortical and striatal progenitor cells;
b) staining for neurofilaments to label axons or dendrites;
c) conducting immunodepletion with neutralizing antibodies; and
d) quantifying axonal or dendritic outgrowth or both.

14. The method of claim 13, wherein the animal model cells are rat cells.

15. The method of claim 13, wherein the neuropotentiating cells are hNPCs.

16. A method of enhancing axonal properties of mammalian brain or spinal tissue, which comprises contacting the tissue with neuropotentiating cell-secreted factors.

17. The method of claim 16, wherein the neuropotentiating cell is hNPC.

18. The method of claim 16, wherein the mammalian brain or spine is human.
